(19) United States
(12) Patent Application Publication
(10) Pub. No.: US 2007/0179123 A1
(43) Pub. Date: Aug. 2, 2007

(54) Methods and compositions for treating diseases associated with pathogenic proteins

(76) Inventors: Peter K. Chiang, San Francisco, CA (US); Daniel E. Levy, San Mateo, CA (US); Meng Guo, Albany, CA (US); Valeri V. Martichonok, San Francisco, CA (US); Brian A. Sefton, Cupertino, CA (US)

Correspondence Address: CYNTHIA RUTH MOORE, 794 LOS ROBLES AVENUE, PALO ALTO, CA, 94306, US

(21) Appl. No.: 11/595,797

(22) Filed: Nov. 8, 2006

Related U.S. Application Data
(60) Provisional application No. 60/735,151, filed on Nov. 8, 2005.
(60) Provisional application No. 60/735,153, filed on Nov. 8, 2005.

(51) Int. Cl. A61K 31/551; A61K 31/502; A61K 31/498; A61K 31/496; A61K 31/4709; A61K 31/4745

(52) U.S. Cl. 514/183; 514/218; 514/210.21; 514/248; 514/249; 514/253.05; 514/253.06; 514/314; 514/319; 514/305; 514/252.17; 514/266.2; 514/266.22

(57) ABSTRACT

Methods and compositions are provided comprising a therapeutically effective amount of a compound of formula I 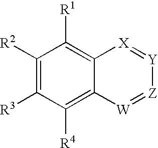
wherein R¹⁻⁴, W, X, Y and Z are as defined in the specification, for inhibiting and treating diseases and disorders associated with pathogenic proteins causing neurodegenerative diseases and amyloid diseases, such as protease resistant prion proteins (PrP^{Sc}) and those associated with transmissible spongiform encephalopathies (TSEs), Alzheimer's Disease, amyloidosis, and the like.

### CROSS-REFERENCE TO RELATED APPLICATIONS

The present application claims priority from U.S. Provisional Patent Application Ser. Nos. 60/735,151 and 60/735,153, both filed on Nov. 8, 2005, which are incorporated by reference herein in their entireties.

### FIELD OF THE INVENTION

The present disclosure relates to compositions for and methods of treatment of diseases associated with pathogenic proteins, such as but not, limited to; amyloid diseases, such as Alzheimer's Disease, protease resistant prion protein (PrP) diseases such as scrapie, transmissible spongiform encephalopathies (TSEs), and disease states where the accumulation, aggregation and/or inherent toxicity of specific proteins are indicated, such as the neurofilaments formed in Amyotropic Lateral Sclerosis, and the like.

### BACKGROUND OF THE INVENTION

Amyloid diseases are diseases or disorders associated with the formation and/or deposition of the pathogenic protein, amyloid. There are many examples of amyloid diseases, and the list includes Alzheimer's disease, systemic senile amyloidosis, prion disease, scrapie, bovine spongiform encephalopathy, Creutzfeldt-Jakob disease, Gerstmann-Straussler-Scheinker syndrome, type I diabetes, adult onset diabetes (type II), insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, lysozyme-associated hereditary systemic amyloidosis, and the like.

Amyloid deposits are found in subjects diagnosed with Alzheimer's disease, a neurodegenerative disease characterized by atrophy of nerve cells in the cerebral cortex, subcortical areas, and hippocampus and the presence of plaques, dystrophic neurites and neurofibrillary tangles. In Alzheimer's disease, dystrophic or aberrant neurite growth, synapse loss, and neurofibrillary tangle formation are strong correlates of disease severity. Dystrophic neurons characteristically contain abundant electrodense multilaminar bodies in the cytoplasm of the neurites and have disruption of synaptic junctions. The dystrophic neurons surround deposits of amyloid, thereby forming the senile plaques located throughout the brain neuropil as well as in the walls of cerebral blood vessels. An efficacious treatment for Alzheimer's disease is needed that could block or reduce the atrophy of nerve cells, or the formation of senile plaques or neurofibrillary tangles, such that the development of the disease is slowed or arrested.

Amyloid deposits are also found in the islets of Langerhans in patients diagnosed with type II diabetes. The deposits contain an amyloid protein that is derived from a larger precursor called islet amyloid polypeptide (IAPP) or amylin which, in normal animals, has a hormonal role. IAPP is produced by the β-cells of the islets and has a profound effect on glucose uptake by the liver and striated muscle cells. In transgenic mice having a transgene for human amylin and which are fed a high fat diet, overproduction of amylin leads to islet amyloid deposition (see Pathology, 3^{rd} ed. (1999) supra, p. 1226). An efficacious treatment for diabetes type II is needed that could reduce or prevent the formation of amyloid deposits in the islets of Langerhans.

Still another disease where amyloid deposits are noted is amyloid A amyloidosis, in which seemingly unrelated disorders such as chronic inflammatory disorders, neoplastic disorders, and hereditary disorders are linked by a common etiology. The deposition of amyloid protein is secondary to the underlying disease condition. The precursor molecule is serum amyloid A (SAA), an acute phase reactant, which can be used as a surrogate marker of inflammation in many diseases. An efficacious treatment for amyloid A amyloidosis is needed that could reduce or prevent the production of amyloid protein, reduce or prevent the production of the precursor to amyloid protein, prevent or reduce any one of several steps necessary to generate an active amyloid protein, or reduce or prevent the deposition of amyloid plaques, or all of the above.

Yet another disease where amyloid deposits are noted is familial transthyretin amyloidosis, which is the most common form of Familial Amyloidotic Polyneuropathy (FAP). The human amyloid disorders, familial amyloid polyneuropathy, familial amyloid cardiomyopathy and senile systemic amyloidosis, are caused by insoluble transthyretin (TTR) fibrils, which deposit in the peripheral nerves and heart tissue. Transthyretin is a homotetrameric plasma protein implicated in the transport of thyroxine and retinol. The most common amyloidogenic TTR variant is V30M-TTR, while L55P-TTR is the variant associated with the most aggressive form of FAP. An efficacious treatment for amyloidoses caused by transthyretin is needed that could reduce or prevent the production of amyloid protein, reduce or prevent the production of the precursor to amyloid protein, prevent or reduce any one of several steps necessary to generate an active amyloid protein, or reduce or prevent the deposition of amyloid plaques, or all of the above.

A further disease where amyloid deposits are noted is AL amyloidosis, which is a class of diseases related to a primary disorder of immunoglobulin production, including primary amyloidosis, plasma cell dyscrasia, immunoblastic lymphoma, multiple myeloma, and the like. Primary systemic AL (amyloid light-chain) amyloidosis is a plasma cell disorder in which depositions of amyloid light-chain protein cause progressive organ failure. The prognosis of primary amyloidosis is generally poor, with a median survival of 1 to 2 years. The precursor protein in both localized and systemic AL-amyloidosis is an immunoglobulin light chain which shows the same pattern of fragmentation and changes of primary structure. An efficacious treatment for amyloidoses caused by AL amyloid proteins is needed that can reduce or prevent the production of amyloid protein, reduce or prevent the production of the precursor to amyloid protein, prevent or reduce any one of several steps necessary to generate an active amyloid protein, or reduce or prevent the deposition of amyloid plaques, or all of the above.

Yet another disease where amyloid deposits are noted is prion disease, one type of spongiform encephalopathy. Prion diseases are neurodegenerative conditions characterized clinically by progressive ataxia and dementia, and pathologically by vacuolization of spongiform brain tissue. Amyloid deposits are associated with at least one prion disease known as kuru. In kuru, about 70% of prion protein accumulates extracellularly to form plaques, in contrast to normal prion protein which is a constitutively expressed cell-surface glycoprotein (see Pathology, 3^{rd} ed. supra, pp. 1492 1496).

Prion diseases show a common and unique post-translational conversion of a normal-host encoded prion protein (PrP^{C}), to an abnormal disease-causing isoform (PrP^{Sc}), which replicates by stimulating the conversion of PrP^{C} into nascent PrP^{Sc}. The latter is a highly aggregated and detergent insoluble polymer very resistant to proteolysis that can form amyloid. The prion diseases or transmissible spongiform encephalopathies (TSEs) are fatal neurodegenerative diseases in humans and some mammalian species. Non-limiting examples of human prion diseases include Gerstmann-Straussler-Scheinker (GSS) disease, fatal insomnia, and Creutzfeldt-Jakob disease (CJD), and kuru.

The endemics and epidemics of prion diseases in animals in the wild and in the meat industry constitute a serious health problem to the public. Bovine spongiform encephalopathy (BSE) is epidemic in some European countries, and a growing epidemic of chronic wasting disease now afflict deer and elk in North America, while scrapie has been endemic in sheep. The outbreak of a new form of CJD (vCJD) among British youngsters, presumably due to dietary exposure to BSE-contaminated beef, points to a new scenario in the transmission of the deadly prion diseases (Silveira et al. (2004) Curr. Topics. Microbiol. Immunol. 284, 1-50).

Treating TSE patients remains a daunting task as there is no therapeutic intervention available and the diseases are uniformly fatal. An efficacious treatment for prion disease is needed that could reduce or prevent the production of amyloid protein, or the deposition of amyloid plaques.

### SUMMARY OF THE INVENTION

Compounds are provided that are useful for preventing, ameliorating, and reversing the effects of diseases and disorders associated with pathogenic proteins, such as amyloid diseases. The compounds include substituted quinolines of formula I 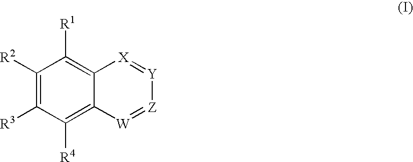
wherein R¹ is selected from the group consisting of H, Cl, Br, I, NO, NO₂, CN, C₁-C₆ alkyl, L¹-R¹⁸ or CF₃; R² is selected from the group consisting of H, Cl, Br, I, NO, NO₂, CN, C₁-C₆ alkyl, L²-(C₁-C₆ alkyl) or CF₃; R³ is selected from the group consisting of H, Cl, Br, I, NO, NO₂, CN, C₁-C₆ alkyl, L³-(C₁-C₆ alkyl) or CF₃; R⁴ is selected from the group consisting of H, Cl, Br, I, NO, NO₂, CN, C₁-C₆ alkyl, L⁴-(C₁-C₆ alkyl), L⁴-aryl or CF₃, and each aryl is optionally substituted with 0-3 groups independently selected from F, Cl, Br, I, OR²⁹ NH₂, CO₂H, CF₃, CN, C₁-C₆ alkyl; R⁵ is selected from the group consisting of H, L⁵-R³² or C(R⁷)₂R²⁸; R⁶ is selected from the group consisting of H, Cl, Br, I, C₁-C₆ alkyl, L⁶-R⁸, CF₃ or aryl, and each aryl is optionally substituted with 0-3 groups independently selected from F, Cl, Br, I, OH, NH₂, CO₂H, CF₃, CN, C₁-C₆ alkyl; R⁷ is independently selected from the group consisting of H, F, C₁-C₆ alkyl, OR⁹, SR¹⁰, S(═O)R¹¹, S(═O)₂R¹² or NR¹³R¹⁴; R⁸ is selected from the group consisting of H or C₁-C₆ alkyl; R⁹ is selected from the group consisting of H or C₁-C₆ alkyl; R¹⁰ is selected from the group consisting of H or C₁-C₆ alkyl; R¹¹ is C₁-C₆ alkyl; R¹² is selected from the group consisting of OH, C₁-C₆ alkyl or O—(C₁-C₆ alkyl); R¹³ is selected from the group consisting of H, C₁-C₆ alkyl or (C═O)—(C₁-C₆ alkyl); R¹⁴ is selected from the group consisting of H, C₁-C₆ alkyl or (C═O)—(C₁-C₆ alkyl); R¹⁵ is selected from the group consisting of H, C₁-C₆ alkyl or (C═O)—(C₁-C₆ alkyl); R¹⁶ is selected from the group consisting of R⁶ or aryl, and each aryl is optionally substituted with 0-3 groups independently selected from F, Cl, Br, I, OH, NH₂, CO₂H, CF₃, CN, C₁-C₆ alkyl; R¹⁷ is selected from the group consisting of H, C₁-C₆ alkyl or aryl, and each aryl is optionally substituted with 0-3 groups independently selected from F, Cl, Br, I, OH, NH₂, CO₂H, CF₃, C₁-C₆ alkyl; R¹⁸ is H, C₁-C₆ alkyl or —(CH₂)ₙCR¹⁹R²⁰(CH₂)ₒ CR³⁰R³¹(CH₂)ₚ—R²¹; R¹⁹ is selected from the group consisting of H, C₁-C₆ alkyl, OH or O—(C₁-C₆ alkyl); R²⁰ is selected from the group consisting of H, C₁-C₆ alkyl, OH or O—(C₁-C₆ alkyl); R²¹ is selected from the group consisting of H, C(═NH)NH₂, OR²², SR²³, S(═O)R²⁴, S(═O)₂R²⁵ or NR²⁶R²⁷; R²² is selected from the group consisting of H or C₁-C₆ alkyl; R²³ is selected from the group consisting of H or C₁-C₆ alkyl; R²⁴ is C₁-C₆ alkyl; R²⁵ is selected from the group consisting of OH, C₁-C₆ alkyl or O—(C₁-C₆ alkyl); R²⁶ is selected from the group consisting of H, (CH₂)_{q}OH, C(═NH)NH₂, C₁-C₈ alkyl or (C═O)—(C₁-C₆ alkyl) where both R²⁶ and R²⁷ are not simultaneously OH and are not simultaneously C(═NH)NH₂; R²⁷ is selected from the group consisting of H, (CH₂)ᵣOH, C(═NH)NH₂, C₁-C₈ alkyl or (C═O)—(C₁-C₆ alkyl) where both R²⁶ and R²⁷ are not simultaneously OH and are not simultaneously C(═NH)NH₂; R²⁸ is selected from the group consisting of H, F and a cyclic amine selected from the following 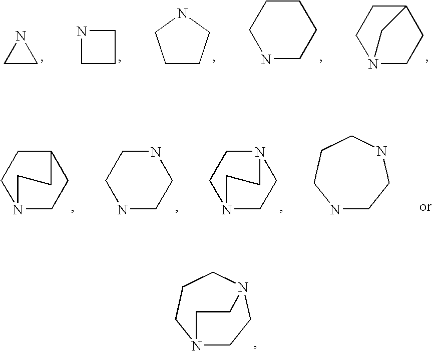
where the attachment point is at any available carbon or nitrogen atom, and all available carbon or nitrogen atoms are optionally substituted with a group independently selected from H, C₁-C₆ alkyl, C₁-C₆ alkenyl, C₁-C₆ alkynyl and (C═O)—(C₁-C₆ alkyl), where no more than two substitutions are not H; R²⁹ is selected from the group consisting of H and C₁-C₆ alkyl; R³⁰ is selected from the group consisting of H and C₁-C₆ alkyl; R³¹ is selected from the group consisting of H and C₁-C₆ alkyl; R³² is either C₁-C₆ alkyl or (CH₂)ₛR²¹; L¹, L², L³, L⁴, L⁵ and L⁶ are independently selected from the group consisting of O, S, S(═O), S(═O)₂ and NR¹⁵; W is N or CR⁵; X is N or CR¹⁶; Y is N or CR⁶; Z is N or CR¹⁷; n is an integer from 0-10; o is an integer from 0-10; p is an integer from 0-10; n+o+p is an integer from 0-12; q is an integer from 0-4; r is an integer from 0-4; and s is either 0 or an integer from 2-6; and pharmaceutically acceptable salts, hydrates, and polymorphs thereof.

As disclosed herein, compounds of formula I have been discovered by Applicant to be inhibitors of the formation of pathogenic proteins, such as prions, PrP^{Sc} and amyloid fibrils, and as such, can be used to treat pathogenic protein diseases. In some embodiments, methods are provided for reducing and/or clearing pathogenic forms of proteins from cells, by contacting the cells with a therapeutically effective amount of a compound of formula I.

Disclosed herein are methods of treating a disease characterized by pathogenic protein formation, by administering a pharmaceutically effective amount of a compound of formula I. Disclosed herein are pharmaceutical compositions comprising a compound of formula I and a pharmaceutically acceptable carrier or diluent, and optionally at least one additional active agent. An additional pharmaceutically active agent is selected from one or more of a cholinomimetic, a cholinesterase inhibitor, a secretase inhibitor, an NMDA receptor antagonist, an amyloid binding protein, insulin or an insulin mimetic, an insulin sensitizer, a PPARγ receptor antagonist, colchicine, an anti-inflammatory agent, GSK-3 inhibitor, levodopa, a dopamine receptor agonist, a catechol —O-methyl transferase inhibitor, a MAO-B inhibitor, a muscarinic receptor antagonist, diphenylhydramine HCl, riluzol, baclofen, tizanidine, clonazepam, or dantroline, and the like.

These methods and compositions are useful in the treatment of a disease characterized by pathogenic protein formation such as Creutzfeldt-Jakob disease, scrapie, transmissible spongiform encephalopathy (TSE), Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy, AIDS dementia, systemic senile amyloidosis, prion disease, Gerstmann-Straussler-Scheinker syndrome, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, or lysozyme-associated hereditary systemic amyloidosis.

In additional aspects, methods for reducing or retarding degeneration in a cell population of a tissue or organ subject to damage in an amyloid disease are provided, comprising exposing the cell population to a therapeutically effective amount of a compound of formula I, and optionally an additional active agent. In an alternative aspect, methods for reducing necrosis or apoptosis in a cell population of a tissue or organ subject to necrotic or apoptotic damage in an amyloid disease are provided, comprising exposing the cell population to a therapeutically effective amount of a compound of formula I, and optionally an additional active agent. In particularly preferred embodiments, the cell population comprises pancreatic islet cells, neurons, glia, endothelial cells, or endocrine cells.

In another aspect, methods of blocking amyloid toxicity in cells are provided, comprising contacting said cells with an effective amount of at least one compound of formula I, and optionally an additional active agent. Preferably, the amyloid toxicity is selected from amyloid beta peptide toxicity, amyloid prion protein toxicity, human amylin toxicity, amyloid A protein toxicity, transthyretin toxicity, and AL amyloid toxicity.

In some embodiments, treatment is by administering a therapeutically effective amount of the pharmaceutical composition to an animal that has been exposed to and/or is in danger of being exposed to the transmissible agent, such as PrP^{Sc}, or which is exhibiting signs, symptoms or laboratory evidence of a TSE. If the animal is merely suspected of having been exposed to a TSE, the treatment is a prophylactic method of preventing the progression of the disease. In a situation where the animal is already believed to be exhibiting signs or symptoms of the disease, the treatment is also a method of improving the neurological or other biological condition of the animal.

In some aspects of the present disclosure, an animal in need of treatment is identified, and a pharmacologically effective amount of a compound of formula I is administered to the animal in an amount sufficient to interfere with PrP^{Sc} formation or accumulation in cells.

The present disclosure includes still other aspects comprising treating an animal, including a mammal, such as a human, having a condition associated with PrP^{Sc}. In this aspect, such a mammal is identified and treated with a compound of formula I, and optionally an additional active agent, in a manner, such as that described herein.

In some aspects of the present disclosure, there is provided a method for reducing or retarding neurodegeneration in a cell population comprising neurons which have been exposed to an amount of a stimulus sufficient to produce partially protease resistant proteins resulting in neurodegeneration; said method comprising administering an efficacious dose of a compound of formula I, and optionally an additional active agent. In some embodiments, the cell population may reside in the central nervous system of an animal and a compound of formula I is administered in vivo. The disclosure also provides the use of a compound of formula I to treat neurodegenerative disease pathology in an animal. In a preferred embodiment, a method is provided for slowing, arresting, or reversing the development of a neurodegenerative disease in a human patient, the method comprising: administering a therapeutically effective amount of a compound of formula I, and optionally an additional active agent, to the patient to inhibit progression of the disease over a therapeutically effective period of time.

Also provided herein are methods for inhibition of neuronal cell death in a cell population. The methods comprise delivering an effective dosage of a compound of formula I to a cell population that is exposed to a protease resistant prion protein stimulus.

In an additional aspect, kits are provided for use in treating a disease or disorder associated with pathogenic proteins comprising a container containing a pharmaceutical composition comprising a therapeutically effective amount of a compound of formula I, and optionally an additional active agent, and instructions comprising guidelines for administration of the composition. Preferably, the instructions comprise an indication of the quantities of the composition to be administered. The kit can further comprise an additional pharmaceutical composition comprising an active agent useful in treating the disease or disorder.

In some aspects of the disclosure, there are provided methods in which a compound of formula I can be combined with a pharmaceutical product; in a particular a product derived from a human source such as organs, tissue, blood, and related blood derived products. In certain other aspects of the disclosure, there are provided methods of treating tissue, organs, blood and blood derived products by combining such with a compound of formula I.

In some aspects of the disclosure, there are provided combinations of livestock feed with a compound of formula I. Non-limiting examples of such feed include meat, bone meal or any material derived from an animal that might be infected with prions.

In certain other aspects of the disclosure, there are provided methods of treating farm animals by administering to farm animals a compound of formula I combined with livestock feed thereby preventing prion infections and/or treating prion diseases such as “mad cow” disease in animals consuming the treated livestock feed.

### BRIEF DESCRIPTION OF THE FIGURES

FIGS. 1A and B show the chemical structures of tafenoquine and mefloquin, respectively.

FIG. 2 demonstrates the effect of tafenoquine (TF) on PrP^{Sc} in RML-infected (10) and 22L-infected (12) N2a cells after 5 days.

FIG. 3 demonstrates the effect of mefloquine (MF) on PrP^{Sc} in RML-infected (●) and 22L-infected (▪) N2a cells after 5 days.

FIG. 4 demonstrates the effect of Compound 3 on PrP^{Sc} in RML-infected (10) and 22L-infected (12) N2a cells after 5 days.

FIG. 5 demonstrates the dose-dependent inhibition of yeast Sup-NM amyloid polymerization by DMSO (20), and by tafenoquine at the following concentrations (μg/mL): 50 (24), 100 (22), 150 (28), and 200 (26).

FIG. 6 demonstrates the dose dependent inhibition of yeast Sup-NM amyloid polymerization by tafenoquine.

FIG. 7 demonstrates the dose dependent inhibition of yeast Sup-NM amyloid polymerization by tafenoquine.

FIG. 8 demonstrates the inhibition of PSI⁺ induction in yeast cells by DMSO (white bars), and tafenoquine (black bars).

FIG. 9 demonstrates the dose dependent inhibition of PSI⁺ induction by tafenoquine.

FIG. 10 demonstrates the dose-dependent inhibition of yeast Sup-NM amyloid polymerization by DMSO (20), and by mefloquine at the following concentrations (μg/mL): 50 (24), 100 (22), 150 (28), and 200 (26).

FIG. 11 demonstrates the concentration dependent inhibition of yeast Sup-NM amyloid polymerization by mefloquin.

FIG. 12 demonstrates the inhibition of PSI⁺ induction in yeast cells by DMSO (white bars), and mefloquin (black bars).

FIG. 13 demonstrates the concentration dependent inhibition of yeast Sup-NM amyloid polymerization by compound 3.

FIG. 14 demonstrates the concentration dependent inhibition of amyloid fibrillogenesis by compound 8.

FIG. 15 demonstrates the concentration dependent inhibition of amyloid fibrillogenesis by compound 8.

FIG. 16 demonstrates the concentration dependent inhibition of fibrillogenesis by mefloquin.

FIG. 17 demonstrates the concentration dependent inhibition of fibrillogenesis by mefloquin.

FIG. 18 demonstrates the concentration dependent inhibition of fibrillogenesis by compound 3.

FIG. 19 demonstrates the concentration dependent inhibition of fibrillogenesis by compound 3.

### DETAILED DESCRIPTION

I. Definitions and Overview

Before the present compositions and methods are described, it is to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

Unless defined otherwise, all technical and scientific terms used herein have the same meaning as commonly understood by one of ordinary skill in the art to which the methods and compositions as disclosed herein belongs. Although any methods and materials similar or equivalent to those described herein can also be used in the practice or testing of the present methods, the preferred methods and materials are now described.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

It must be noted that as used herein and in the appended claims, the singular forms “a,” “and” and “the” include plural referents unless the context clearly dictates otherwise. Thus, for example, reference to “a compound” includes a plurality of such compounds and reference to “the step” includes reference to one or more steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided might be different from the actual publication dates, which may need to be independently confirmed.

The term “pathogenic” is used in its conventional sense, and refers to the quality of causing disease or of being capable of causing disease (Merriam-Webster's Medical Desk Dictionary, 1996).

The term “pathogenic protein” refers to proteins that are malformed (i.e., insoluble or denatured), misprocessed, toxic, infectious, or that otherwise exhibit a deleterious effect on health and/or biological function. Such proteins may have residual activity or no activity, but in all cases are associated with disease and pathogenesis in the affected animal or human patient.

As used herein, formula I encompasses compounds of formulas II, III, IV and V. Thus, reference to compounds of formula I is intended to refer to compounds of formulas II, III, IV and V as well, unless clearly indicated otherwise.

The pharmaceutically acceptable acid addition salts of compounds of the formula I, II, III, IV and/or V may be used in the various compositions and methods described herein. The compounds of formula I, II, III, IV and V are basic in nature and are thus capable of forming a wide variety of salts with various inorganic and organic acids. The acids that may be used to prepare pharmaceutically acceptable acid addition salts of those compounds of formula I, II, III, IV and V are those that form non-toxic acid addition salts, i.e., salts containing pharmacologically acceptable anions, such as, but not limited to, the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, fumarate, gluconate, glucaronate, saccharate, formate, benzoate, glutamate, methanesulfonate, ethanesulfonate, benzenesulfonate, and p-toluenesulfonate. Compounds of formula I, II, III, IV and V may be converted into pharmaceutically acceptable salts by methods well known to those skilled in the art.

The term “one or more substituents”, as used herein, includes from one to the maximum number of substituents possible based on the number of available bonding sites.

The term “therapeutically effective amount,” as used herein, refers to an amount sufficient to detectably treat, ameliorate, prevent or retard the progression of an unwanted condition or symptom associated with disorders related to pathogenic proteins. In some instances, the amount is that determined to provide an in vitro effect sufficient to treat, ameliorate, prevent or detectably retard the progression of an unwanted condition or symptom.

The chemist of ordinary skill will recognize that certain combinations of substituents included within the scope of formulas I, II, III, IV and/or V may be chemically unstable and will avoid these combinations or alternatively protect sensitive groups with well known protecting groups.

The term “alkyl”, as used herein, unless otherwise indicated, includes saturated monovalent hydrocarbon radicals with 1 to 12 carbon atoms having straight, branched or cyclic moieties or combinations thereof. The term “lower alkyl” refers to an alkyl group having one to six carbon atoms. Examples include methyl, ethyl, propyl, isopropyl, cyclopropyl, butyl, isobutyl, sec-butyl, t-butyl, pentyl, neopentyl, cyclopentylmethyl, and hexyl. It is preferred that the alkyl group is lower alkyl.

The term “alkenyl” as used herein refers to a hydrocarbon radical with two to eight carbon atoms and at least one double bond. The alkenyl group may be straight-chained, branched, or cyclic, and may be in either the Z or E form. Examples include ethenyl, 1-propenyl, 2-propenyl, 1-butenyl, 2-butenyl, isopropenyl, isobutenyl, 1-pentenyl, (Z)-2-pentenyl, (E)-2-pentenyl, (Z)-4-methyl-2-pentenyl, (E)-4-methyl-2-pentenyl, 1,3-pentadienyl, 2,4-pentadienyl, 1,3-butadienyl, cyclopentadienyl, and the like.

The term “alkynyl” refers to a hydrocarbon radical with two to eight carbon atoms and at least one carbon-carbon triple bond. The alkynyl group may be straight chained or branched. Examples include 1-propynyl, 2-propynyl, 1-butynyl, 2-butynyl, 1-pentynyl, 2-pentynyl, 3-methyl-1-pentynyl, 3-pentynyl, 1-hexynyl, 2-hexynyl, 3-hexynyl, and the like.

The term “aryl”, as used herein, unless otherwise indicated, includes an organic radical derived from a C₆-C₁₄ aromatic hydrocarbon by removal of one or more hydrogen(s), such as, but not limited to, phenyl and naphthyl. The term aryl also includes “heteroaryl” which, unless otherwise indicated, indicating an organic radical derived from a C₅-C₁₄ aromatic heterocyclic compound by removal of one or more hydrogens. Nonlimiting examples of heteroaryl groups include benzimidazolyl, benzofuranyl, benzofurazanyl, 2H-1-benzopyranyl, benzothiadiazine, benzothiazinyl, benzothiazolyl, benothiophenyl, benzoxazolyl, furazanyl, furopyridinyl, furyl, imidazolyl, indazolyl, indolinyl, indolizinyl, indolyl, 3H-indolyl, isiondolyl, isoquinolinyl, isothiazolyl, isoxazolyl, naphthyridinyl, oxadiazolyl, oxazolyl, phthalazinyl, pteridinyl, purinyl, pyrazinyl, pyridazinyl, pyridinyl or pyridyl, pyrimidinyl, pyrazolyl, pyrrolyl, quinazolinyl, quinolinyl, quinoxalinyl, tetrazolyl, thiazolyl, thiadiazolyl, thienyl, triazinyl and triazolyl. Preferred aryl groups include 5 or 6 membered rings containing 0-4 heteroatoms selected from O, N, and S and optionally substituted with 0-4 groups independently selected from F, Cl, Br, I, hydroxyl, alkoxy, NH₂, CO₂H, CF₃, CN, or C₁-C₆ alkyl. For example, preferred “aryl” substituents include, but are not limited to, phenyl, pyridyl, pyrimidyl, furyl, thienyl, oxazolyl, thiazolyl or imidazolyl.

Unless indicated otherwise, compounds of the present invention include all stereoisomers, R and S configurations at stereogenic centers, as well as E and Z configurations around carbon carbon double bonds.

The terms “treatment,” “treating,” “treat” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in an animal, particularly a human, and includes: (a) preventing the disease or symptom from occurring in a subject which may be predisposed to the disease or symptom but has not yet been diagnosed as having it; (b) inhibiting the disease of its symptom, i.e., arresting development of the disease or its symptoms; or (c) relieving the disease symptom, i.e., causing regression of the disease or symptom.

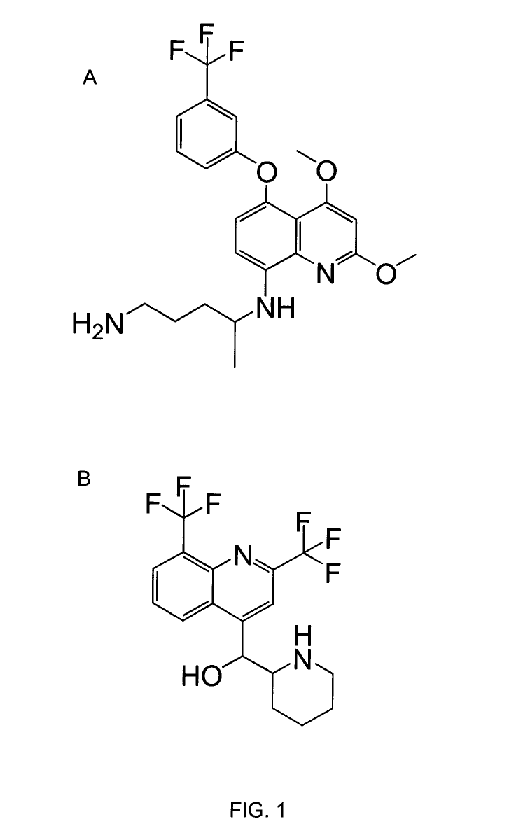

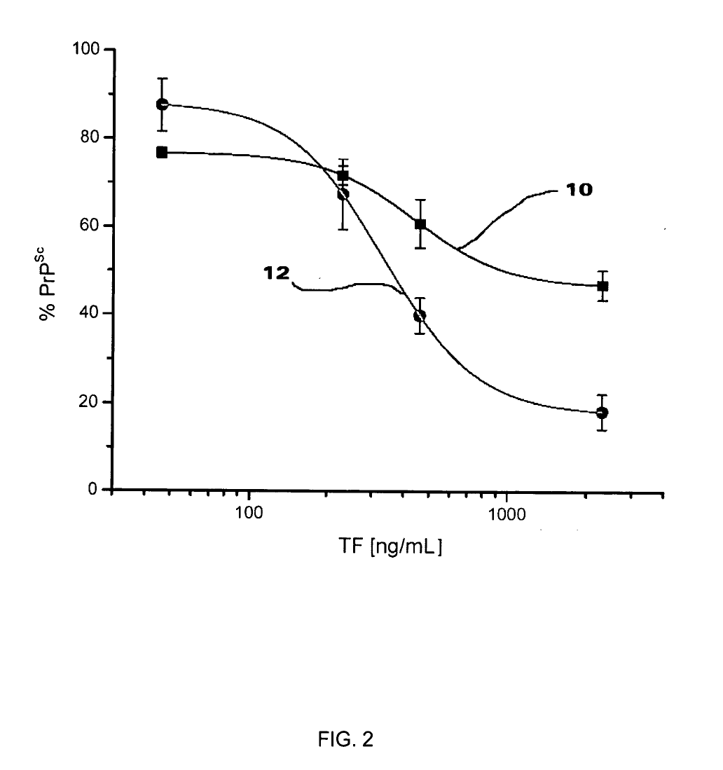

Tafenoquine [(±)-8-[(4-amino-1-methylbutyl) amino]-2,6-dimethoxy-4-methyl-5-(3-trifluoromethylphenoxy)quinoline] and mefloquine [DL-erythro-α-2-piperidyl-2,8-bis(trifluoromethyl)-4-quinolinemethanol] (FIG. 1) have been shown to be very suitable for the treatment of malaria, especially of tropical malaria (see, e.g., Peters, et al. (1993) Ann. Trop. Med. Parasitol. 87, 547-552) and U.S. Pat. No. 4,696,921). Applicants have surprisingly discovered that compounds of formula I such as tafenoquine, mefloquin and certain compounds shown in Table 1 can significantly reduce the amount of PrP^{Sc} in N2a cells that were infected with two different strains of mouse-adapted scrapie prions, RML and 22L (see Examples 1 to 3). For example, tafenoquine, compound 3 and mefloquin reduced PrP^{Sc} concentration from N2a cells in a dose-dependent manner. See Examples 1 to 3 and FIGS. 2-4. Accordingly, an animal suffering from a condition associated with pathogenic PrP^{Sc} formation and accumulation can be treated by administration of a composition of formula I, optionally comprising an additional active agent, as disclosed herein.

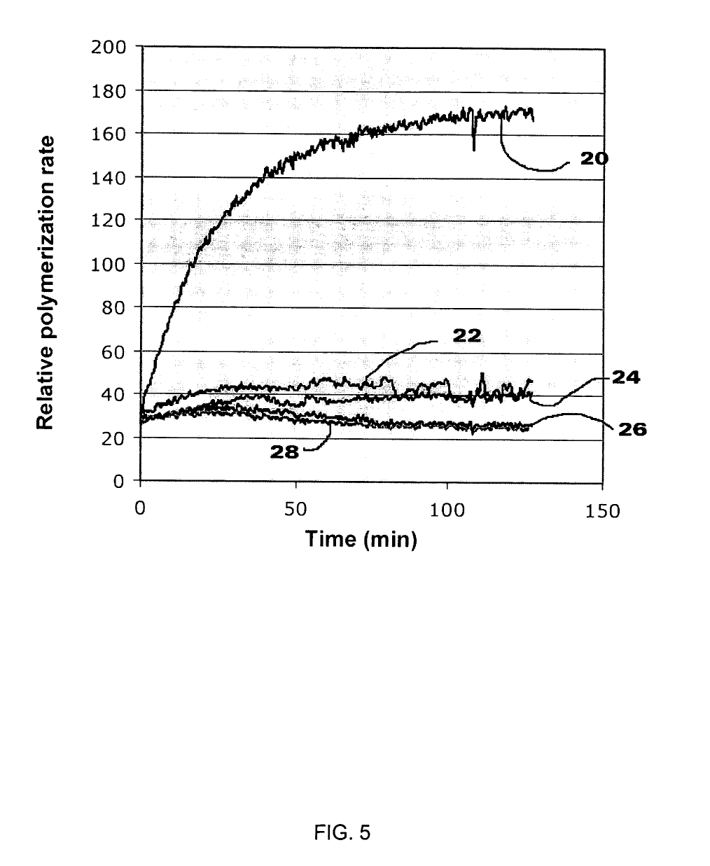

Applicants have also surprisingly demonstrated a significant reduction in amyloid formation by tafenoquine and other compounds of formula I in a yeast cell model system (see Examples 4 to 6 and FIGS. 5-13). The transmissible dementias or spongiform encephalopathies such as Creutzfeldt-Jakob disease, Gerstmann-Straussler syndrome, kuru and a variety of other diseases such as scrapie, are all characterized by abnormal accumulations of PrP^{Sc}. As such, an animal suffering from such accumulation can be effectively treated by administration of a composition of formula I, optionally comprising an additional active agent, as disclosed herein.

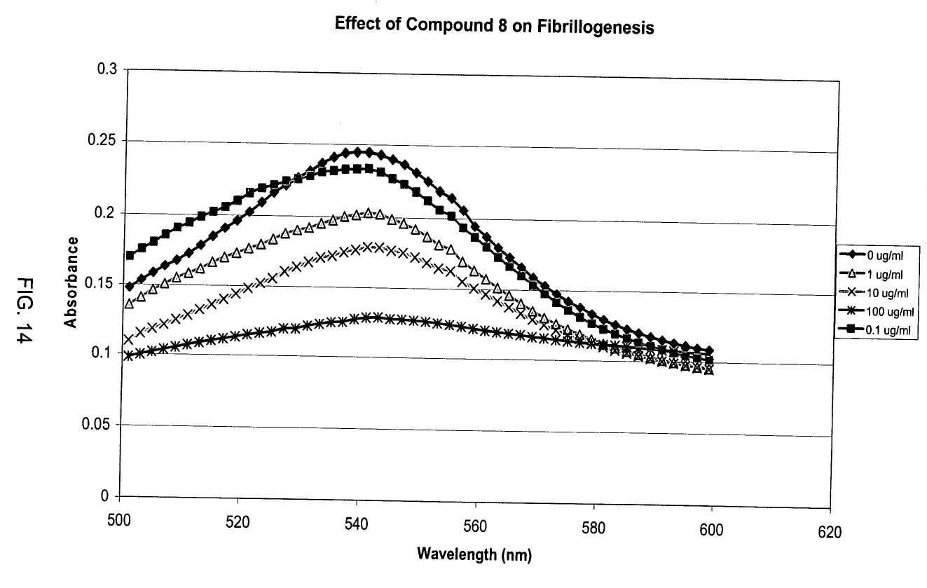

Applicants have further surprisingly discovered that compounds of formula I are effective to reduce or reverse amyloid fibril formation (fibrillogenesis). As shown in Examples 7 to 9 and FIGS. 14-19, compounds of formula I were effective to reduce the ability of amyloid peptides in solution to form fibrils, and to cause an increase in the amount of amyloid peptide monomer after incubation of compounds with amyloid fibrils. Accordingly, an animal suffering from a condition associated with pathogenic amyloid fibril formation can be treated by administration of a composition of formula I, optionally comprising an additional active agent, as disclosed herein.

II. Compounds

Compounds disclosed herein are useful for preventing, ameliorating, and reversing the effects of diseases and disorders associated with pathogenic proteins, such as amyloid diseases. The compounds include substituted quinolines of formula I 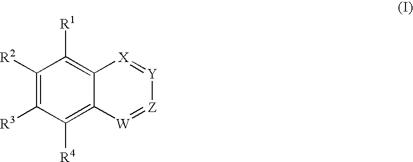
wherein R¹ is selected from the group consisting of H, Cl, Br, I, NO, NO₂, CN, C₁-C₆ alkyl, L¹-R¹⁸ or CF₃; R² is selected from the group consisting of H, Cl, Br, I, NO, NO₂, CN, C₁-C₆ alkyl, L²-(C₁-C₆ alkyl) or CF₃; R³ is selected from the group consisting of H, Cl, Br, I, NO, NO₂, CN, C₁-C₆ alkyl, L³-(C₁-C₆ alkyl) or CF₃; R⁴ is selected from the group consisting of H, Cl, Br, I, NO, NO₂, CN, C₁-C₆ alkyl, L⁴-(C₁-C₆ alkyl), L⁴-aryl or CF₃, and each aryl is optionally substituted with 0-3 groups independently selected from F, Cl, Br, I, OR²⁹ NH₂, CO₂H, CF₃, CN, C₁-C₆ alkyl; R⁵ is selected from the group consisting of H, L⁵-R³² or C(R⁷)₂R²⁸; R⁶ is selected from the group consisting of H, Cl, Br, I, C₁-C₆ alkyl, L⁶-R⁸, CF₃ or aryl, and each aryl is optionally substituted with 0-3 groups independently selected from F, Cl, Br, I, OH, NH₂, CO₂H, CF₃, CN, C₁-C₆ alkyl; R⁷ is independently selected from the group consisting of H, F, C₁-C₆ alkyl, OR⁹, SR¹⁰, S(═O)R¹¹, S(═O)₂R¹² or NR¹³R¹⁴; R⁸ is selected from the group consisting of H or C₁-C₆ alkyl; R⁹ is selected from the group consisting of H or C₁-C₆ alkyl; R¹⁰ is selected from the group consisting of H or C₁-C₆ alkyl; R¹¹ is C₁-C₆ alkyl; R¹² is selected from the group consisting of OH, C₁-C₆ alkyl or O—(C₁-C₆ alkyl); R¹³ is selected from the group consisting of H, C₁-C₆ alkyl or (C═O)—(C₁-C₆ alkyl); R¹⁴ is selected from the group consisting of H, C₁-C₆ alkyl or (C═O)—(C₁-C₆ alkyl); R¹⁵ is selected from the group consisting of H, C₁-C₆ alkyl or (C═O)—(C₁-C₆ alkyl); R¹⁶ is selected from the group consisting of R⁶ or aryl, and each aryl is optionally substituted with 0-3 groups independently selected from F, Cl, Br, I, OH, NH₂, CO₂H, CF₃, CN, C₁-C₆ alkyl; R¹⁷ is selected from the group consisting of H, C₁-C₆ alkyl or aryl, and each aryl is optionally substituted with 0-3 groups independently selected from F, Cl, Br, I, OH, NH₂, CO₂H, CF₃, CN, C₁-C₆ alkyl; R¹⁸ is H, C₁-C₆ alkyl or —(CH₂)ₙCR¹⁹R²⁰(CH₂)ₒ CR³⁰R³¹(CH₂)ₚ—R²¹; R¹⁹ is selected from the group consisting of H, C₁-C₆ alkyl, OH or O—(C₁-C₆ alkyl); R²⁰ is selected from the group consisting of H, C₁-C₆ alkyl, OH or O—(C₁-C₆ alkyl); R²¹ is selected from the group consisting of H, C(═NH)NH₂, OR²², SR²³, S(═O)R²⁴, S(═O)₂R²⁵ or NR²⁶R²⁷; R²² is selected from the group consisting of H or C₁-C₆ alkyl; R²³ is selected from the group consisting of H or C₁-C₆ alkyl; R²⁴ is C₁-C₆ alkyl; R²⁵ is selected from the group consisting of OH, C₁-C₆ alkyl or O—(C₁-C₆ alkyl); R²⁶ is selected from the group consisting of H, (CH₂)_{q}OH, C(═NH)NH₂, C₁-C₈ alkyl or (C═O)—(C₁-C₆ alkyl) where both R²⁶ and R²⁷ are not simultaneously OH and are not simultaneously C(═NH)NH₂; R²⁷ is selected from the group consisting of H, (CH₂)ᵣOH, C(═NH)NH₂, C₁-C₈ alkyl or (C═O)—(C₁-C₆ alkyl) where both R²⁶ and R²⁷ are not simultaneously OH and are not simultaneously C(═NH)NH₂; R²⁸ is selected from the group consisting of H, F and a cyclic amine selected from the following 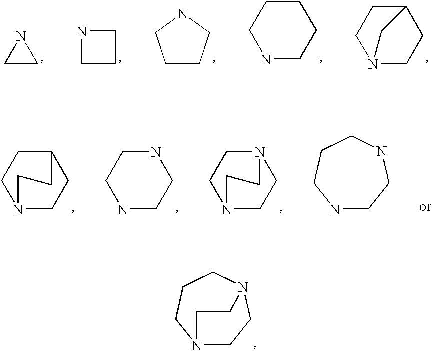
where the attachment point is at any available carbon or nitrogen atom, and all available carbon or nitrogen atoms are optionally substituted with a group independently selected from H, C₁-C₆ alkyl, C₁-C₆ alkenyl, C₁-C₆ alkynyl and (C═O)—(C₁-C₆ alkyl), where no more than two substitutions are not H; R²⁹ is selected from the group consisting of H and C₁-C₆ alkyl; R³⁰ is selected from the group consisting of H and C₁-C₆ alkyl; R³¹ is selected from the group consisting of H and C₁-C₆ alkyl; R³² is either C₁-C₆ alkyl or (CH₂)ₛR²¹; L¹, L², L³, L⁴, L⁵ and L⁶ are independently selected from the group consisting of O, S, S(═O), S(═O)₂ and NR¹⁵; W is N or CR⁵; X is N or CR¹⁶; Y is N or CR⁶; Z is N or CR¹⁷; n is an integer from 0-10; o is an integer from 0-10; p is an integer from 0-10; n+o+p is an integer from 0-12; q is an integer from 0-4; r is an integer from 0-4; and s is either 0 or an integer from 2-6; and pharmaceutically acceptable salts, hydrates, and polymorphs thereof.

In a preferred embodiment, the compounds include substituted quinolines of formula II 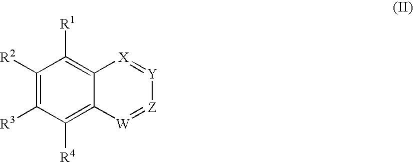
wherein R¹ is selected from the group consisting of H, Cl, Br, I, NO, NO₂, C₁-C₆ alkyl, L¹-R¹⁸ or CF₃; R² is selected from the group consisting of H, Cl, Br, I, NO, NO₂, C₁-C₆ alkyl, L²-(C₁-C₆ alkyl) or CF₃; R³ is selected from the group consisting of H, Cl, Br, I, NO, NO₂, C₁-C₆ alkyl, L³-(C₁-C₆ alkyl) or CF₃; R⁴ is selected from the group consisting of H, Cl, Br, I, NO, NO₂, C₁-C₆ alkyl, L⁴-(C₁-C₆ alkyl), L⁴-aryl or CF₃ and each aryl is optionally substituted with 0-3 groups independently selected from F, Cl, Br, I, OR²⁹ NH₂, CO₂H, CF₃, C₁-C₆ alkyl; R⁵ is H, L⁵-R³² or C(R⁷)₂R²⁸; R⁶ is selected from the group consisting of H, Cl, Br, I, C₁-C₆ alkyl, L⁶-R⁸ or CF₃; R⁷ is independently selected from the group consisting of H, F, C₁-C₆ alkyl, OR⁹, SR¹⁰, S(═O)R¹¹, S(═O)₂R¹² or NR¹³R¹⁴; R⁸ is selected from the group consisting of H or C₁-C₆ alkyl; R⁹ is selected from the group consisting of H or C₁-C₆ alkyl; R¹⁰ is selected from the group consisting of H or C₁-C₆ alkyl; R¹¹ is C₁-C₆ alkyl; R¹² is selected from the group consisting of OH, C₁-C₆ alkyl or O—(C₁-C₆ alkyl); R¹³ is selected from the group consisting of H, C₁-C₆ alkyl or (C═O)—(C₁-C₆ alkyl); R¹⁴ is selected from the group consisting of H, C₁-C₆ alkyl or (C═O)—(C₁-C₆ alkyl); R¹⁵ is selected from the group consisting of H, C₁-C₆ alkyl or (C═O)—(C₁-C₆ alkyl); R¹⁷ is selected from the group consisting of H or C₁-C₆ alkyl; R¹⁸ is H, C₁-C₆ alkyl or —(CH₂)ₙCR¹⁹R²⁰(CH₂)ₒ CR³⁰R³¹(CH₂)ₚ—R²¹; R¹⁹ is selected from the group consisting of H, C₁-C₆ alkyl, OH or O—(C₁-C₆ alkyl); R²⁰ is selected from the group consisting of H, C₁-C₆ alkyl, OH or O—(C₁-C₆ alkyl); R²¹ is selected from the group consisting of H, C(═NH)NH₂, OR²², SR²³, S(═O)R²⁴, S(═O)₂R²⁵ or NR²⁶R²⁷; R²² is selected from the group consisting of H or C₁-C₆ alkyl; R²³ is selected from the group consisting of H or C₁-C₆ alkyl; R²⁴ is C₁-C₆ alkyl; R²⁵ is selected from the group consisting of OH, C₁-C₆ alkyl or O—(C₁-C₆ alkyl); R²⁶ is selected from the group consisting of H, (CH₂)_{q}OH, C(═NH)NH₂, C₁-C₈ alkyl or (C═O)—(C₁-C₆ alkyl), where R²⁶ and R²⁷ are not both simultaneously OH or C(═NH)NH₂; R²⁷ is selected from the group consisting of H, (CH₂)ᵣOH, C(═NH)NH₂, C₁-C₈ alkyl or (C═O)—(C₁-C₆ alkyl), where R²⁶ and R²⁷ are not both simultaneously OH or C(═NH)NH₂; R²⁸ is selected from the group consisting of H, F, and a cyclic amine selected from the following 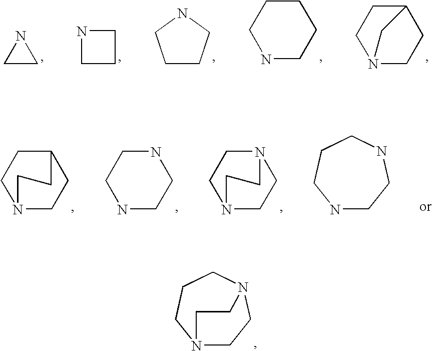
where the attachment point is at any available carbon or nitrogen atom, and all available carbon or nitrogen atoms are additionally substituted with groups independently selected from H, C₁-C₆ alkyl, C₁-C₆ alkenyl, C₁-C₆ alkynyl or (C═O)—(C₁-C₆ alkyl), where no more than two substitutions are not H; R²⁹ is selected from the group consisting of H or C₁-C₆ alkyl; R³⁰ is selected from the group consisting of H or C₁-C₆ alkyl; R³¹ is selected from the group consisting of H or C₁-C₆ alkyl; R³² is either C₁-C₆ alkyl or (CH₂)ₛR²¹; L¹, L², L³, L⁴, L⁵ and L⁶ are independently selected from the group consisting of O, S, S(═O), S(═O)₂ or NR¹⁵; W is N or CR⁵; X is N or CR⁶; Y is N or CR⁶; Z is N or CR¹⁷; n is an integer from 0-10; o is an integer from 0-10; p is an integer from 0-10; n+o+p is an integer from 0-12; q is an integer from 0-4; r is an integer from 0-4; s is either 0 or an integer from 2-6; and pharmaceutically acceptable salts, hydrates, and polymorphs thereof.

In an additional preferred embodiment, the compounds include substituted quinolines of formula III 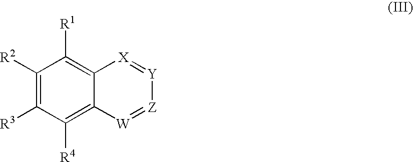
wherein R¹ is selected from the group consisting of H, Cl, Br, I, NO, NO₂, C₁-C₆ alkyl, L¹-R¹⁸ or CF₃; R² is selected from the group consisting of H, Cl, Br, I, NO, NO₂, C₁-C₆ alkyl, L²-(C₁-C₆ alkyl) or CF₃; R³ is selected from the group consisting of H, Cl, Br, I, NO, NO₂, C₁-C₆ alkyl, L³-(C₁-C₆ alkyl) or CF₃; R⁴ is selected from the group consisting of H, Cl, Br, I, NO, NO₂, C₁-C₆ alkyl, L⁴-(C₁-C₆ alkyl), L⁴-aryl or CF₃ and each aryl is optionally substituted with 0-3 groups independently selected from F, Cl, Br, I, OR²⁹ NH₂, CO₂H, CF₃, C₁-C₆ alkyl; R⁵ is H, L⁵-R³² or C(R⁷)₂R²⁸; R⁶ is selected from the group consisting of H, Cl, Br, I, C₁-C₆ alkyl, L⁶-R⁸ or CF₃; R⁷ is independently selected from the group consisting of H, F, C₁-C₆ alkyl, OR⁹, SR¹⁰ or NR¹³R¹⁴; R⁸ is selected from the group consisting of H or C₁-C₆ alkyl; R⁹ is selected from the group consisting of H or C₁-C₆ alkyl; R¹⁰ is selected from the group consisting of H or C₁-C₆ alkyl; R¹³ is selected from the group consisting of H, C₁-C₆ alkyl or (C═O)—(C₁-C₆ alkyl); R¹⁴ is selected from the group consisting of H, C₁-C₆ alkyl or (C═O)—(C₁-C₆ alkyl); R¹⁵ is selected from the group consisting of H, C₁-C₆ alkyl or (C═O)—(C₁-C₆ alkyl); R¹⁷ is selected from the group consisting of H or C₁-C₆ alkyl; R¹⁸ is H, C₁-C₆ alkyl or —(CH₂)ₙCR¹⁹R²⁰(CH₂)ₒ CR³⁰R³¹ (CH₂)ₚ—R²; R¹⁹ is selected from the group consisting of H, C₁-C₆ alkyl, OH or O—(C₁-C₆ alkyl); R²⁰ is selected from the group consisting of H, C₁-C₆ alkyl, OH or O—(C₁-C₆ alkyl); R²¹ is selected from the group consisting of H, C(═NH)NH₂, OR²², SR²³ or NR²⁶R²⁷; R²² is selected from the group consisting of H or C₁-C₆ alkyl; R²³ is selected from the group consisting of H or C₁-C₆ alkyl; R²⁶ is selected from the group consisting of H, (CH₂)_{q}OH, C(═NH)NH₂, C₁-C₈ alkyl or (C═O)—(C₁-C₆ alkyl) where R²⁶ and R²⁷ are not both simultaneously OH or C(═NH)NH₂; R²⁷ is selected from the group consisting of H, (CH₂)ᵣOH, C(═NH)NH₂, C₁-C₈ alkyl or (C═O)—(C₁-C₆ alkyl), where R²⁶ and R²⁷ are not both simultaneously OH or C(═NH)NH₂; R²⁸ is selected from the group consisting of H, F and a cyclic amine selected from the following 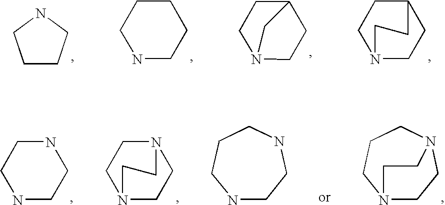
where the attachment point is at any available carbon or nitrogen atom, and all available carbon or nitrogen atoms are additionally substituted with groups independently selected from H, C₁-C₆ alkyl, C₁-C₆ alkenyl, C₁-C₆ alkynyl or (C═O)—(C₁-C₆ alkyl), where no more than two substitutions are not H; R²⁹ is selected from the group consisting of H or C₁-C₆ alkyl; R³⁰ is selected from the group consisting of H or C₁-C₆ alkyl; R³¹ is selected from the group consisting of H or C₁-C₆ alkyl; R³² is either C₁-C₆ alkyl or (CH₂)ₛR²¹; L¹, L², L³, L⁴, L⁵ and L⁶ are independently selected from the group consisting of O, S or NR¹⁵; W is N or CR⁵; X is N or CR⁶; Y is N or CR⁶; Z is N or CR¹⁷; n is an integer from 0-10; o is an integer from 0-10; p is an integer from 0-10; n+o+p is an integer from 0-12; q is an integer from 0-4; r is an integer from 0-4; s is either 0 or an integer from 2-6; and pharmaceutically acceptable salts, hydrates, and polymorphs thereof.

In a particularly preferred embodiment, the compounds include substituted quinolines of formula IV 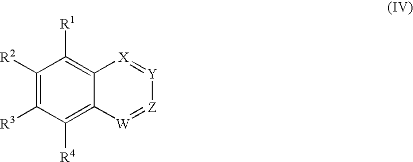
wherein R¹ is selected from the group consisting of H, C₁-C₆ alkyl, L¹-R¹⁸ or CF₃; R² is selected from the group consisting of H, C₁-C₆ alkyl, L²-(C₁-C₆ alkyl) or CF₃; R³ is selected from the group consisting of H, C₁-C₆ alkyl, L³-(C₁-C₆ alkyl) or CF₃; R⁴ is selected from the group consisting of H, C₁-C₆ alkyl, L⁴-(C₁-C₆ alkyl) or CF₃; R⁵ is C(R⁷)₂R²⁸; R⁶ is selected from the group consisting of H, C₁-C₆ alkyl, L⁶-R⁸ or CF₃; R⁷ is independently selected from the group consisting of H or OR⁹; R⁸ is C₁-C₆ alkyl; R⁹ is selected from the group consisting of H or C₁-C₆ alkyl; R¹⁵ is selected from the group consisting of H, C₁-C₆ alkyl or (C═O)—(C₁-C₆ alkyl; R¹⁷ is selected from the group consisting of H or C₁-C₆ alkyl; R¹⁸ is H or C₁-C₆ alkyl; R²⁸ is selected from a cyclic amine selected from the following

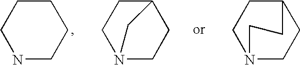

where the attachment point is at any available carbon or nitrogen atom, and all available carbon or nitrogen atoms are additionally substituted with groups independently selected from H, C₁-C₆ alkyl, C₁-C₆ alkenyl, C₁-C₆ alkynyl or (C═O)—(C₁-C₆ alkyl), where no more than two substitutions are not H; L¹, L², L³, L⁴ and L⁶ are independently selected from the group consisting of O, S or NR¹⁵; W is CR⁵; X is N; Y is CR⁶; Z is CR¹⁷; and pharmaceutically acceptable salts, hydrates, and polymorphs thereof. In a particularly preferred embodiment, the quinoline compound of formula IV is mefloquin.

In an alternative embodiment, the compounds include substituted quinolines of formula V

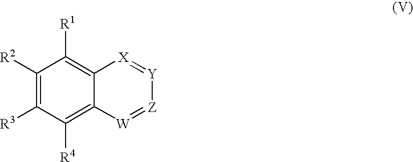

wherein R¹ is selected from the group consisting of H, C₁-C₆ alkyl L¹-R¹⁸ or CF₃; R² is selected from the group consisting of H, C₁-C₆ alkyl L²(C₂-C₆ alkyl) or CF₃; R³ is selected from the group consisting of H, C₁-C₆ alkyl, L³-(C₁-C₆ alkyl) or CF₃; R⁴ is L⁴-aryl and each aryl is optionally substituted with 0-3 groups independently selected from F, Cl, Br, I, OR²⁹ NH₂, CO₂H, CF₃, C₁-C₆ alkyl; R⁵ is H, L⁵-R³² or C(R⁷)₂R²⁸; R⁶ is selected from the group consisting of H, C₁-C₆ alkyl, L⁶-R⁸ or CF₃; R⁷ is H; R⁸ is C₁-C₆ alkyl; R¹⁵ is selected from the group consisting of H, C₁-C₆ alkyl or (C═O)—(C₁-C₆ alkyl); R¹⁷ is H; R¹⁸ is —(CH₂)ₙCR¹⁹R²⁰(CH₂)ₒ CR³⁰R³¹(CH₂)ₚ—R²¹; R¹⁹ is H or C₁-C₆ alkyl; R²⁰ is H or C₁-C₆ alkyl; R²¹ is NR²⁶R²⁷; R²⁶ is H or C₁-C₈ alkyl; R²⁷ is H or C₁-C₈ alkyl; R²⁸ is H; R²⁹ is selected from the group consisting of H or C₁-C₆ alkyl; R³⁰ is selected from the group consisting of H or C₁-C₆ alkyl; R³¹ is selected from the group consisting of H or C₁-C₆ alkyl; R³¹ is C₁-C₆ alkyl; L¹, L², L³, L⁴, L⁵ and L⁶ are independently selected from the group consisting of O, S or NR¹⁵; W is CR⁵; X is N; Y is CR⁶; is CR¹⁷; n is an integer from 0-5; o is an integer from 0-5; p is an integer from 0-5; n+o+p is an integer from 0-5; and pharmaceutically acceptable salts, hydrates, and polymorphs thereof. In particularly preferred embodiments, the quinoline compound of formula V is tafenoquine. In additional embodiments, the quinoline compound is selected from a compound shown in Table I.

TABLE I
Compounds of Formula I.

| | IC₅₀ for | | | IC₅₀ for | |
|---|---|---|---|---|---|
| | purging of | IC₅₀ for | | inhibition of | |
| | PRP^{Sc} in | purging of | IC₅₀ for | Sup35 yeast | IC₅₀ for |
| | RML-infected | PRP^{Sc} in 22L- | inhibition of | prion | inhibition of |
| | N2a cells | infected N2a | induction of | polymerization | fibrillogenesis |
| Structure and ID number | (ng/mL) | cells (ng/mL) | PSI+ (μg/mL) | (μg/mL) | (μg/mL) |
| 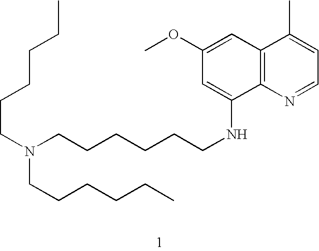 | | | | | <5 |
| 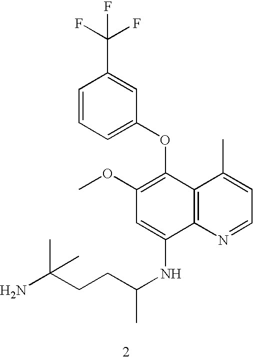 | | | | | 15-30 |
| 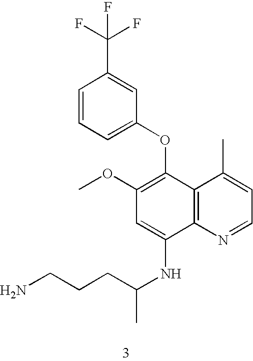 | 264 | 220 | | 15 | 10 |
| 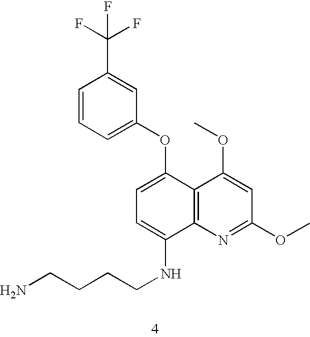 | 332 | 440 | 4.5 | 5.2 ± 1.1 | |
| 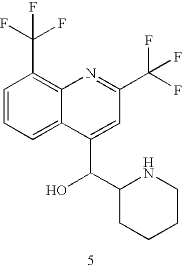 | 224 (0.5 μM) | 482 | | | |
| 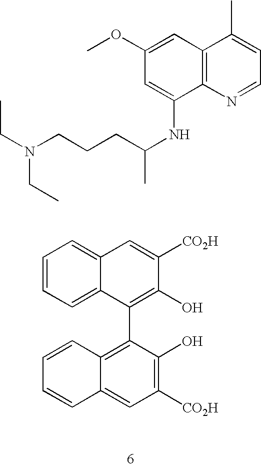 | | (3-5 μM) | | | |
| 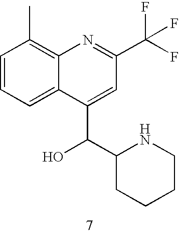 | | | | | |
| 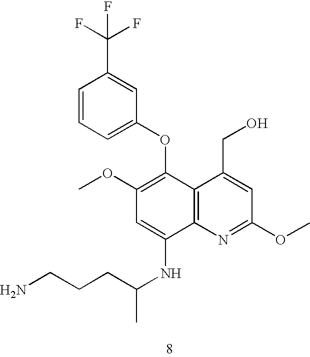 | | | | | ˜5 |
| 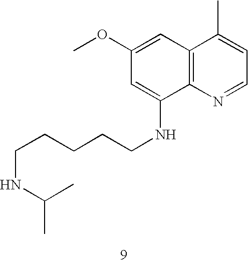 | | | | | ˜5 |
| 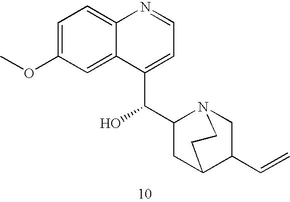 | (6 μM) | | | | |
| 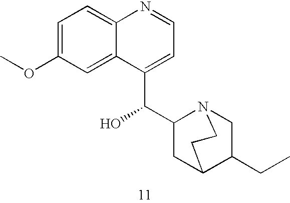 | (12.5 μM) | | | | |
| 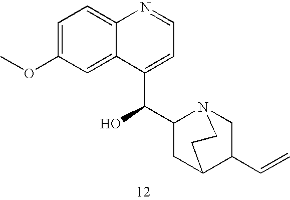 | (3 μM) | | | | |
| 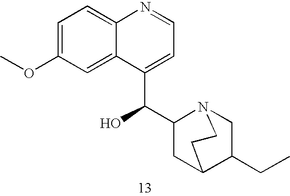 | (toxic @ μM) | | | | |
| 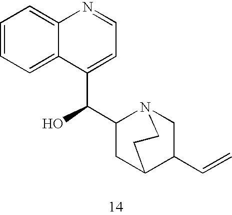 | (6 μM) | | | | |
| 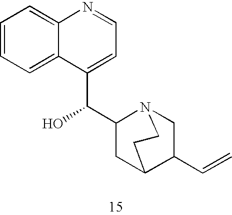 | (18 μM) | | | | |
| 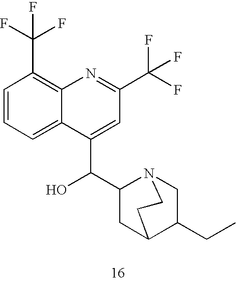 | | | | | |
| 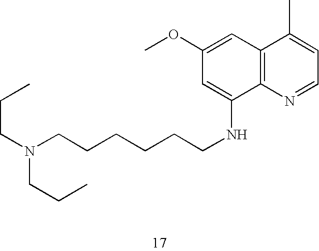 | | | | | |
| 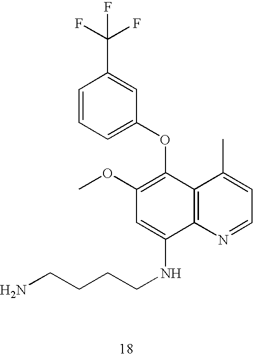 | | | | | |
| 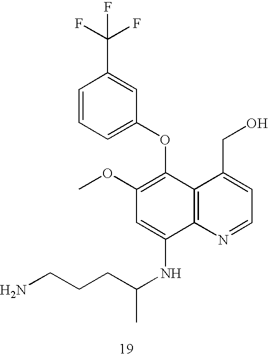 | | | | | |
| 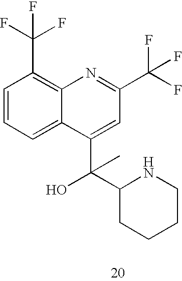 | | | | | |
| 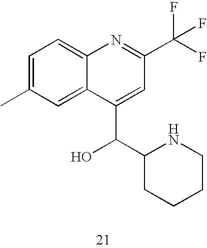 | | | | | |
| 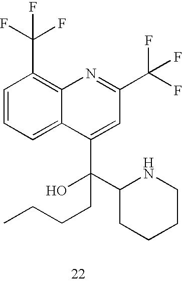 | | | | | |
| 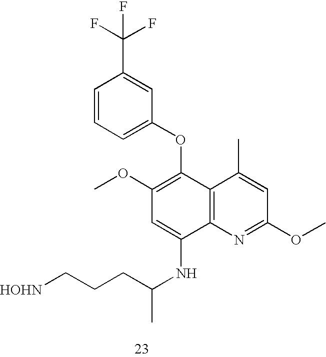 | ˜5 μg/mL | | | | |
| 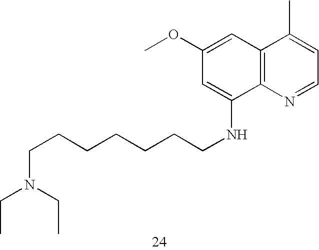 | | | | | |
| 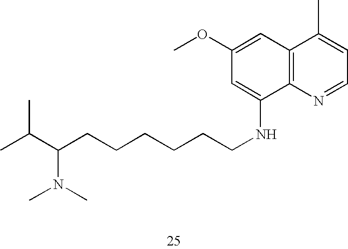 | | | | | |
| 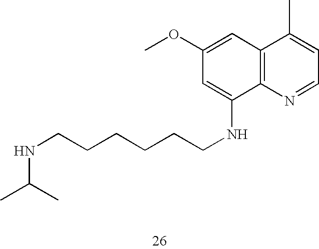 | | | | | |
| 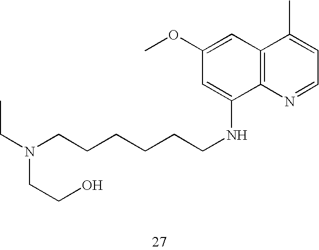 | | | | | |
| 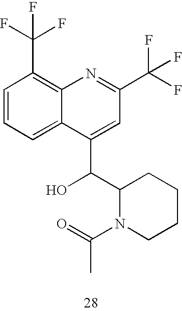 | | | | | |
| 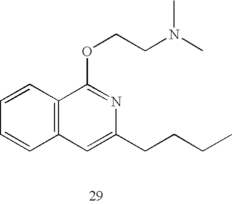 | | | | | |
| 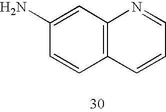 | | | | | |
| 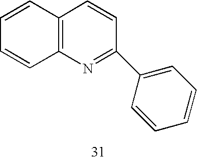 | | | | | |
| 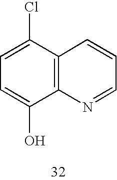 | | Toxic @ 10 μM | | | |
| 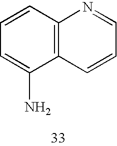 | | | | | |
| 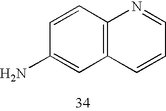 | | | | | |
| 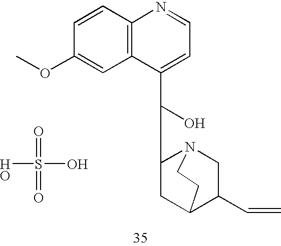 | | (3-5 μM) | | | |
| 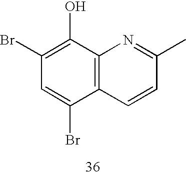 | | | | | |
| 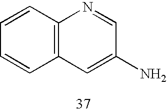 | | | | | |
| 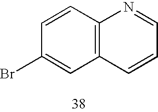 | | | | | |
| 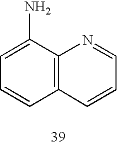 | | | | | |
| 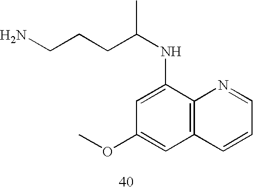 | (<10 μM) | | | | |
| 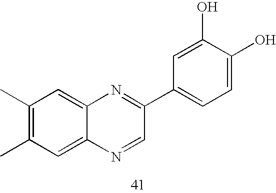 | | | | | |
| 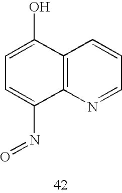 | | | | | |
| 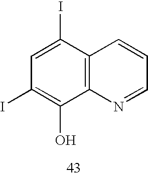 | | | | | |
| 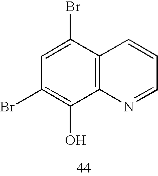 | | | | | |
| 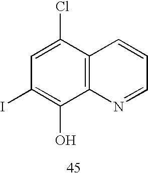 | | | | | |
| 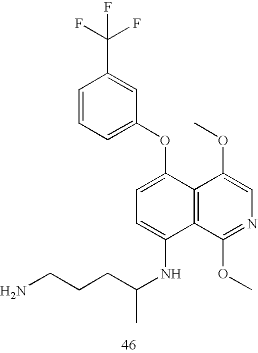 | | | | | |

III. Methods for Preparing Compounds of Formula I

The compounds of formula I can be prepared according to the methods as discussed below. Except where otherwise stated, Ar, W, X, Y, Z, and n in the reaction schemes and discussion that follow are defined as above. Unless otherwise stated reaction conditions include an inert atmosphere commonly used in the art such as nitrogen or argon.

Tafenoquine (TF) can be synthesized using the scheme shown below from commercially available starting materials and reagents. Heating of 4,5-dichloro-2-nitro-phenylamine (SigmaAldrich, 10 g) at 130° C. with 3-oxo-butyric acid ethyl ester produces N-(4,5-dichloro-2-nitro-phenyl)-3-oxo-butyramide which is treated with sulfuric acid at 100° C. to give 5,6-dichloro-4-methyl-8-nitro-quinolin-2-ol. These two reactions are called Knorr quinoline synthesis and their conditions can be found in the literature (1). Treatment of 5,6-dichloro-4-methyl-8-nitro-quinolin-2-ol with sodium hydride and methyl iodide in DMF forms 5,6-dichloro-2-methoxy-4-methyl-8-nitro-quinoline. The chlorine in the para-position to the nitro group in the last compound is more reactive towards a nucleophilic attack than the chlorine in the 6-position. The reaction with 3-trifluoromethyl-phenol gives 6-chloro-2-methoxy-4-methyl-8-nitro-5-(3-trifluoromethyl-phenoxy)-quinoline. Substitution of the chlorine in the 6-position with a methoxy group is achieved by refluxing with sodium methoxide in methanol to produce 2,6-dimethoxy-4-methyl-8-nitro-5-(3-trifluoromethyl-phenoxy)-quinoline which is reduced in a Parr hydrogenator (or alternatively with Zn in acetic acid) to 2,6-dimethoxy-4-methyl-5-(3-trifluoromethyl-phenoxy)-quinolin-8-ylamine. Reductive amination (sodiumcyano borohydride, acetic acid) of 2,6-dimethoxy-4-methyl-8-nitro-5-(3-trifluoromethyl-phenoxy)-quinolin-8-ylamine with (4-oxo-pentyl)-carbamic acid tert-butyl ester gives {4-[2,6-dimethoxy-4-methyl-5-(3-trifluoromethyl-phenoxy)-quinolin-8-ylamino]-pentyl}-carbamic acid tert-butyl ester (not shown in the Scheme) which is treated with HCl in diethyl ether to form the desired TF as an HCl salt (N4-[2,6-dimethoxy-4-methyl-5-(3-trifluoromethyl-phenoxy)-quinolin-8-yl]-pentane-1,4-diamine hydrochloride). Total-8 steps. (4-oxo-pentyl)-Carbamic acid tert-butyl ester (A) used in this synthesis is not commercially available. It can be prepared in 2 steps starting with 4-tert-butoxycarbonyl amino-butyric acid. Reacting 4-tert-butoxycarbonylamino-butyric acid with Weinreb amine produces [3-(methoxy-methyl-carbamoyl)-propyl]-carbamic acid tert butyl ester which is treated in the next step with methylmagnesium bromide to give the desired material A. See C. R. Hauser and G. A. Reynolds, J. Am. Chem Soc. 70, 2402 (1948); Organic Syntheses coll. vol. 111, 593 (New York, 1955).

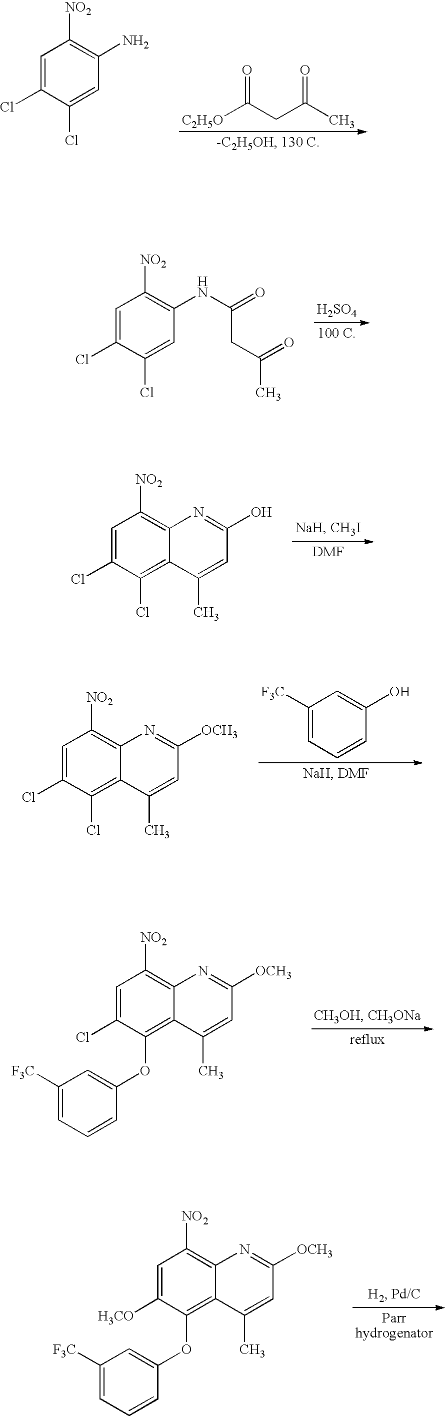

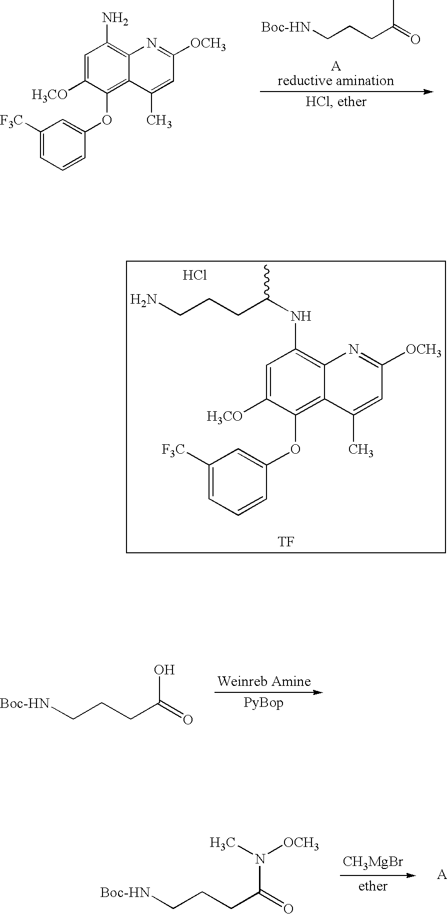

Iso-Tafenoquine (isoTF) can be synthesized using the scheme shown below from commercially available starting materials and reagents. The Friedel-Crafts reaction (1,2,3) of 1,2-dichloro-4-nitro-benzene (available from SigmaAldrich, 100 g) with acetic anhydride produces 1-(2,3-dichloro-5-nitro-phenyl)-ethanone which can then be subjected to a modified Pictet-Gams isoquinoline synthesis conditions (4,5). A reaction of 1-(2,3-dichloro-5-nitro-phenyl)-ethanone with HNO₂ gives 1-(2,3-dichloro-5-nitro-phenyl)-2-nitroso-ethanone, its reduction produces 2-amino-1-(2,3-dichloro-5-nitro-phenyl)-ethanone, a reaction with methylchloroformate forms [2-(2,3-dichloro-5-nitro-phenyl)-2-oxo-ethyl]-carbamic acid methyl ester. Peterson olefination (6,7) gives [2-(2,3-dichloro-5-nitro-phenyl)-allyl]-carbamic acid methyl ester which is cyclized under Picted-Gams conditions to 5,6-dichloro-1-methoxy-4-methyl-8-nitro-isoquinoline. Five steps are required from here to make the desired isoTF (N4-[1,6-dimethoxy-4-methyl-5-(3-trifluoromethyl-phenoxy)-isoquinolin-8-yl]-pentane-1,4-diamine). These 5 steps are the same as for the synthesis of TF (see synthesis of TF).

Briefly, the chlorine in the para-position to the nitro group in the 5,6-dichloro-1-methoxy-4-methyl-8-nitro-isoquinoline is more reactive towards a nucleophilic attack than the chlorine in the 6-position. The reaction with 3-trifluoromethyl-phenol gives 6-chloro-1-methoxy-4-methyl-8-nitro-5-(3-trifluoromethyl-phenoxy)-isoquinoline. Substitution of the chlorine in the 6-position with a methoxy group is achieved by reflux with sodium methoxide in methanol to produce 1,6-dimethoxy-4-methyl-8-nitro-5-(3-trifluoromethyl-phenoxy)-isoquinoline which is reduced in a Parr hydrogenator (or alternatively with Zn in acetic acid) to 1,6-dimethoxy-4-methyl-5-(3-trifluoromethyl-phenoxy)-isoquinolin-8-ylamine. Reductive amination (sodiumcyanoborohydride, acetic acid) of 1,6-dimethoxy-4-methyl-5-(3-trifluoromethyl-phenoxy)-isoquinolin-8-ylamine with (4-oxo-pentyl)-carbamic acid tert-butyl ester gives {4-[1,6-dimethoxy-4-methyl-5-(3-trifluoromethyl-phenoxy)-isoquinolin-8-ylamino]-pentyl}-carbamic acid tert-butyl ester which is treated with HCl in diethyl ether to form the desired isoTF hydrochloride. Total-11 steps. Useful references include: E. Berliner in Organic Reactions V, p 229 (New York, 1949); C. C. Price in Organic Reactions III, p 1 (New York, 1946); W. S. Johnson in Organic Reactions V, p 130 (New York, 1944); W. Hertz and L. Tsai, J. Am. Chem. Soc. 77, 3529 (1955), A. Pictet and A. Gams, Ber. 43, 2384 (1910), S. E. Kelly, Comp. Org. Syn. 1,731-737, 782-783 (1991), and D. J. Ager, Org. React. 38, 1-223 (1990).

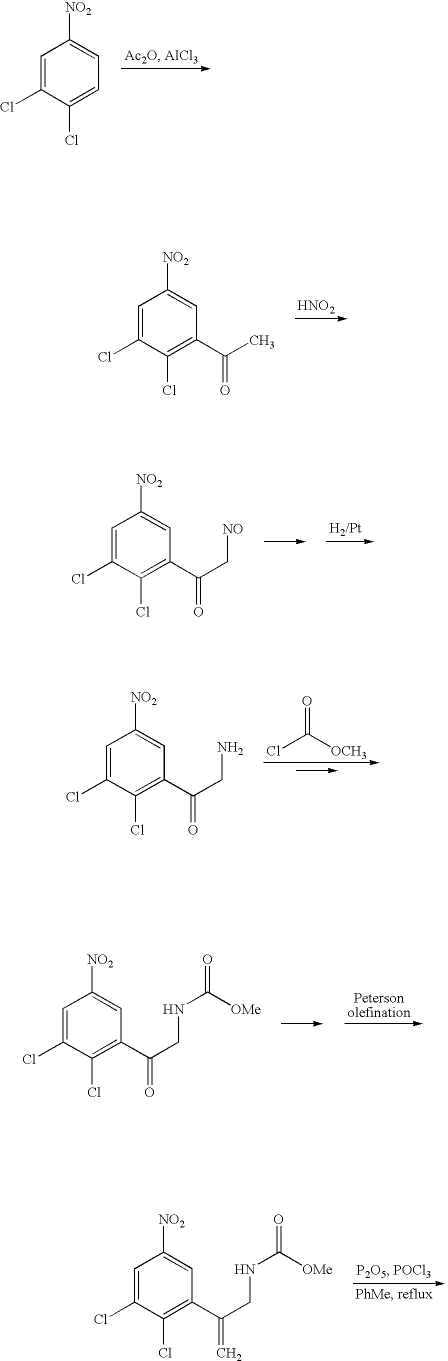

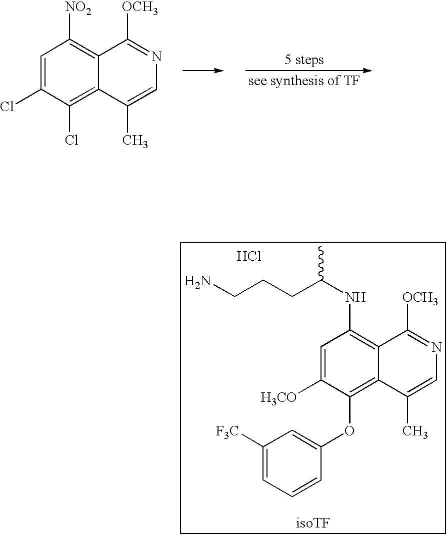

Mefloquine (MF) can be synthesized as shown in the scheme below by condensing 4-Bromo-2,8-bis(trifluoromethyl)quinoline 5 with 2-pyridylacetonitrile 6 in the presence of benzyltriethylammonium chloride in tetrahydrofuran to give nitrile 7 that is oxidized with tert-butyl peroxide to ketone 8. The reduction of 8 with sodium borohydride and the reduction of the pyridyl ring with hydrogen over a palladium catalyst produces racemic MF and analogs 10. 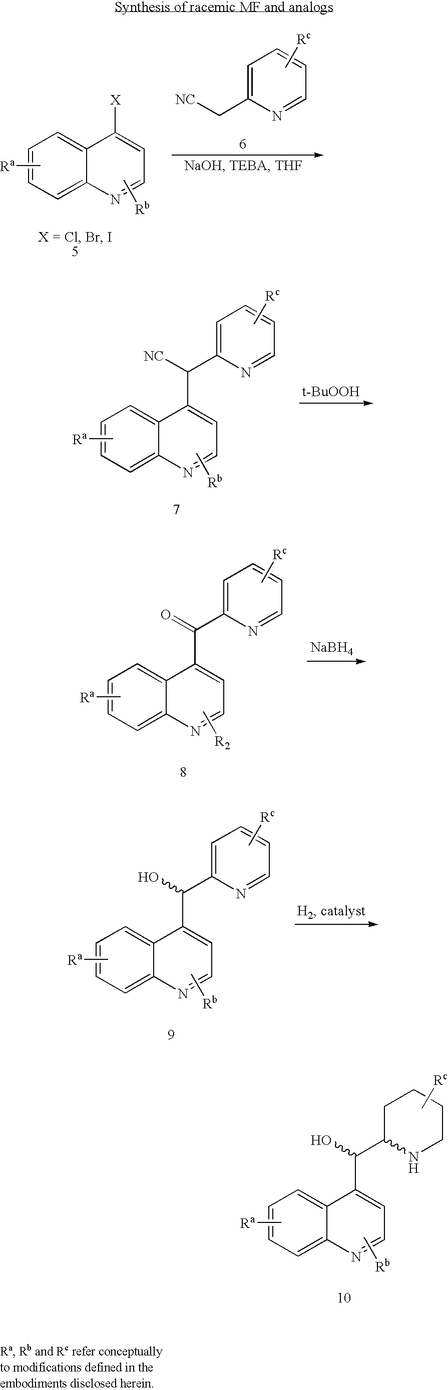
MF is produced as a racemic mixture, and its stereoisomers are shown below.

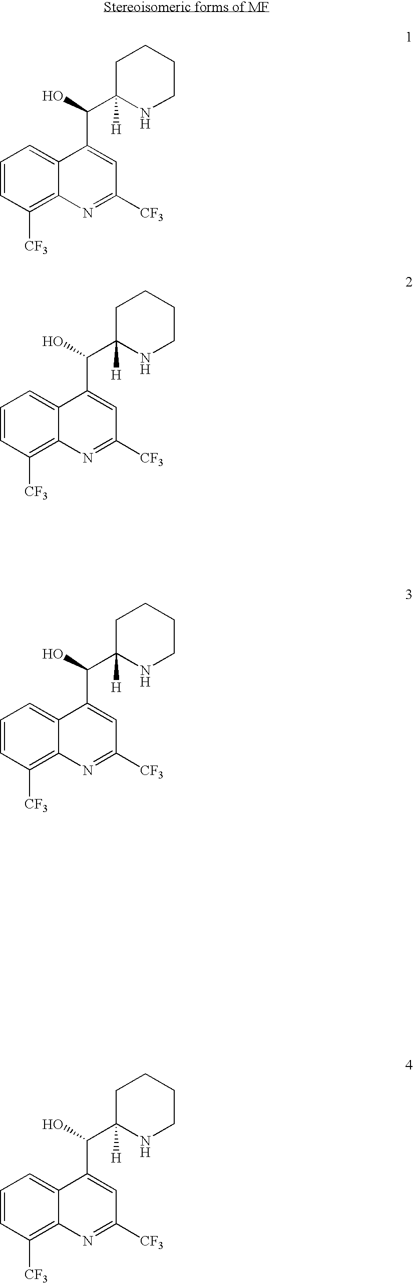

Enantiomerically pure MF can be prepared by a recrystallization of salts of tartaric acid (3). Acylation of the pyridyl intermediate 10 with acetic anhydride yields acetate 11. Treatment with lipase produces a mixture of unreacted enantiomer 12 and desired secondary alcohol 13 which is reduced with hydrogen over a palladium catalyst to MF and analogs 14. Acylation of 10 gives acetamide 15 which is separated using an acylase to give MF analogs 16. Useful references include: Karle, J. M., Karle, I. L. Antimicrob Agents Chemother. 2002; 46 (5), 1529-34; U.S. Patent Application Publication No. 2002188129; December 2002; Carroll F. I., Blackwell, J. T. J Med Chem. 1974; 17 (2), 210-9; Jones, J. B. Tetrahedron (1986); 42, 3351-3403; Koeller, K. M., Wong, C. H. Nature (2001); 409, 232-240; and Enzymes in Synthetic Organic Chemistry, by Chi-Huey Wong, George M. Whitesides 1994.

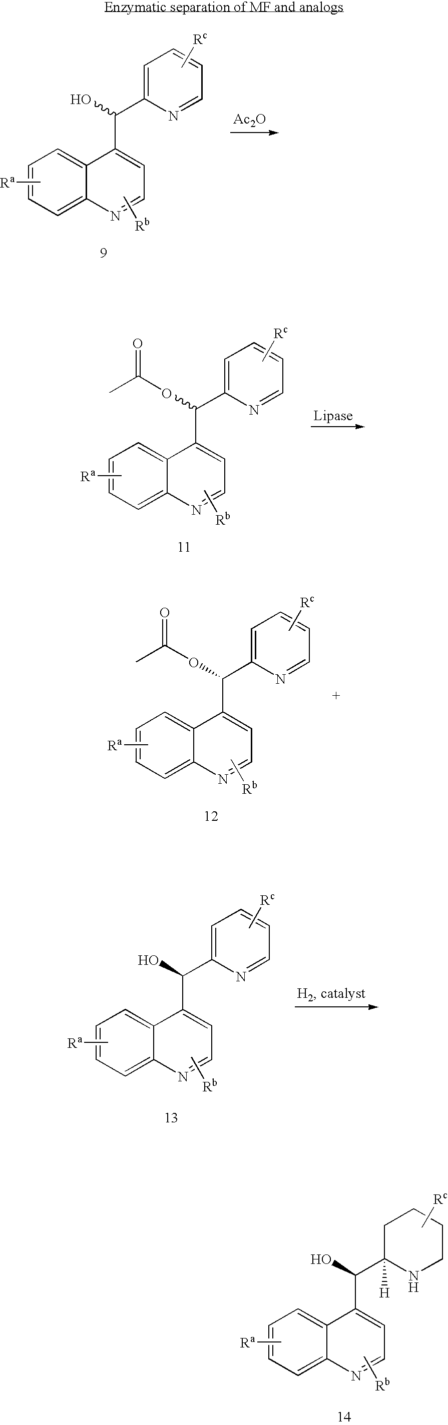

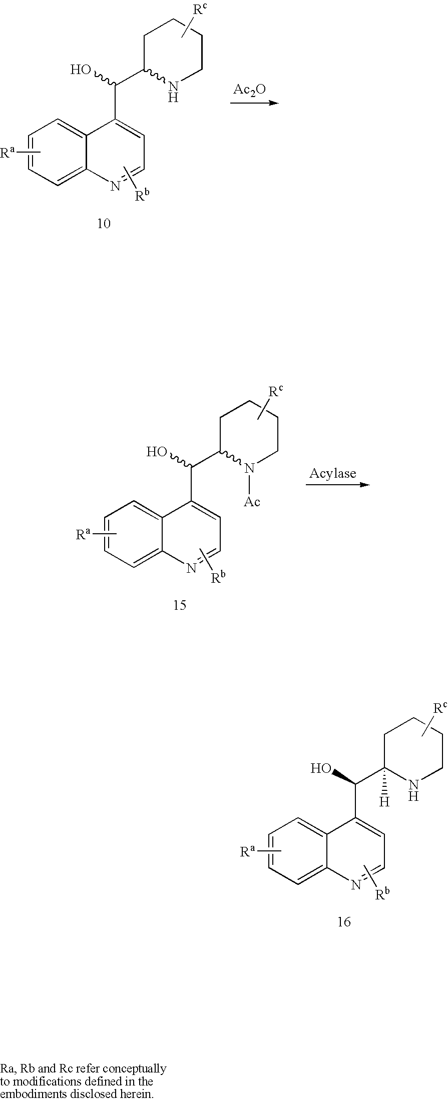

All modifications specified herein are accessible through modifications to the illustrated synthetic schemes readily identified by one skilled in the art. For example, referring to the synthesis of TF, all embodiments of R¹—R⁴ are readily accessible utilizing either commercially available alternatives to 3,4-dichloro-6-nitroaniline or via readily accessible chemical modifications of 3,4-dichloro-6-nitroaniline. Such modifications include, but are not limited to, incorporation of alternate alkylating agents, ketones or aldehydes to effect direct alkylation or reductive amination of the amine precursor to TF. Furthermore, utilizing 3,4-dichloro-6-hydroxyaniline or 3,4-dichloro-6-thioaniline, linker modifications incorporating oxygen or sulfur are accessible. Furthermore, oxidation of sulfide analogs to sulfoxides or sufones is easily achieved using oxidizing agents such as mCPBA or others known to one skilled in the art. Regarding alternate positions, aryl chlorides can be directly displaced with nucleophilic functionalities consistent with the embodiments described herein and are readily recognized by one skilled in the art.

Referring to R⁵, R⁶ and R¹⁷, replacement of the initial methyl ketone of the keto ester with alternate alkyl ketones provides structural diversity at R⁵ consistent with the embodiments described herein. Addition of substitutions at the carbon separating the carbonyls of the initial keto ester provides structural diversity at R¹⁷ consistent with the embodiments described herein. Alkylation of the hydroxy group of the illustrated hydroxyquinoline derivative with alternate alkylating agents provides structural diversity at R⁶ consistent with the embodiments described herein. Alternatively, the hydroxy group of the illustrated hydroxyquinoline derivative is easily converted to a chloride using thionyl chloride or phosphorus oxychloride. The hydroxy group of the illustrated hydroxyquinoline is also easily converted to a triflate using triflic anhydride. The resulting chloroquinoline analogs or quinoline triflates are easily displaced with nucleophilic functionalities consistent with the embodiments described herein and are readily recognized by one skilled in the art. Additionally, the resulting chloroquinoline analogs or quinoline triflates enable incorporation of aryl groups utilizing arylboronic acids, arylboronic esters and arylstannanes coupled with Stille or Suzuki-type methodologies well known to those skilled in the are. Such aryl modifications are consistent with the embodiments described herein.

Regarding R¹⁶, isolation of the intermediate hydroxyisoquinoline (not shown in the isoTF scheme) followed by alkylation with alternate alkylating agents provides structural diversity at R¹⁶ consistent with the embodiments described herein. Furthermore, the hydroxy group of the intermediate hydroxyisoquinoline is easily converted to a chloride using thionyl chloride or phosphorus oxychloride. The hydroxy group of the illustrated hydroxyisoquinoline is also easily converted to a triflate using triflic anhydride. The resulting chloroisoquinoline analogs or isoquinoline triflates are easily displaced with nucleophilic functionalities consistent with the embodiments described herein and are readily recognized by one skilled in the art. Additionally, the resulting chloroisoquinoline analogs or isoquinoline triflates enable incorporation of aryl groups utilizing arylboronic acids, arylboronic esters and arylstannanes coupled with Stille or Suzuki-type methodologies well known to those skilled in the are. Such aryl modifications are consistent with the embodiments described herein.

Hence, all modifications surrounding the core structures of the present invention and described in the embodiments (as R groups, L groups, W, X, Y and Z) presented herein are fully supported using known chemical transformations and techniques well known to those skilled in the art. Further support for incorporation of aryl groups as substituents of the core structures is easily achieved utilizing Suzuki, Stille, Sonagashira or any other transition metal mediated cross-coupling reaction known to one skilled in the art. The following book titles support the breadth of chemistry available to accomplish such modifications: Louis Hegedus, Transition Metals in the Synthesis of Complex Organic Molecules, University Science Books; 2nd edition (Jun. 2, 1999); N. Miyaura, S. L. Buchwald, and K. Fugami, Cross-Coupling Reactions: A Practical Guide (Topics in Current Chemistry), Springer; first edition (Jan. 24, 2006); Armin De Meijere and Francois Diederich, Eds., Metal-Catalyzed Cross-Coupling Reactions, John Wiley & Sons; 2nd Rv&Enl edition (Nov. 10, 2004).

IV. Compositions

Compositions provided herein include pharmaceutically acceptable dosage forms for oral, parenteral, inhalation, transdermal and topical administration, as discussed further below. Such dosage forms can optionally comprise pharmaceutically acceptable carriers, diluents, and the like, and such formulations and formulation methods are well known to those of skill in the art.

Compositions and kits can also include an additional active agent useful for the treatment of diseases and conditions associated with pathogenic proteins described herein. For treatment, amelioration and/or prevention of Alzheimer's Disease, compositions can comprise a pharmaceutically acceptable dosage form comprising a compound of formula I alone or in combination with an additional active agent useful for preventing, ameliorating or reversing Alzheimer's Disease, or for treating the symptoms associated with Alzheimer's Disease. Additional active agents include but are not limited to those selected from a secretase inhibitor, such as a β-secretase inhibitor, including but not limited to compounds described in U.S. Pat. Nos. 6,586,475 to Kato, 7,119,085 to Hom, or a γ-secretase inhibitor; an acetylcholinesterase inhibitor, including but not limited to donepezil (ARICEPT); amyloid binding proteins, such as an antibody having specific binding for amyloid; neuroprotective agents such as the moderate affinity NMDA receptor antagonists, including by not limited to memantine; and other agents typically used in the treatment of patients with Alzheimer's disease such as psychotropics, estrogen, statins, nonsteroidal antiinflammatory agents, and antioxidants.

For treatment, amelioration and/or prevention of diabetes type I, compositions can comprise a pharmaceutically acceptable dosage form comprising a compound of formula I and an additional active agent useful for preventing, ameliorating or reversing diabetes, or alone or in combination with treating the symptoms associated with diabetes. Additional active agents include but are not limited to a glycogen synthase kinase 3 (GSK-3) inhibitor, such as a thiadiazolidinone (e.g., NP031112 (NeuroPharma), insulin or insulin mimetic, insulin sensitizer, such as metformin or the PPAR-γ receptor antagonists, such as the thiazolidinediones (e.g., rosiglitazone (AVANDIA) or pioglitazone (ACTOS, Takeda), or anti-hyperglycemic agents such as glucophage (METFORMIN) or sulfonylureas such as glipizide (GLUCOTROL).

For treatment, amelioration and/or prevention of diabetes type II, compositions can comprise a pharmaceutically acceptable dosage form comprising a compound of formula I alone or in combination with an additional active agent useful for preventing, ameliorating or reversing diabetes, or for treating the symptoms associated with diabetes. Additional active agents include but are not limited to a glycogen synthase kinase 3 (GSK-3) inhibitor, such as a thiadiazolidinone (e.g., NP031112 (NeuroPharma), insulin or insulin mimetic, insulin sensitizer, such as metformin or the PPAR-γ receptor antagonists, such as the thiazolidinediones (e.g., rosiglitazone (AVANDIA) or pioglitazone (ACTOS, Takeda), or anti-hyperglycemic agents such as glucophage (METFORMIN) or sulfonylureas such as glipizide (GLUCOTROL); and insulin or an insulin mimetic.

For treatment, amelioration and/or prevention of Parkinson's disease, compositions can comprise a pharmaceutically acceptable dosage form comprising a compound of formula I alone or in combination with an additional active agent useful for preventing, ameliorating or reversing Parkinson's disease, or for treating the symptoms associated with Parkinson's disease. Additional active agents include but are not limited to levodopa, dopamine receptor agonists, such as bromocriptine; catechol —O-methyl transferase (COMT) inhibitors such as tolcapon; MAO-B inhibitors, such as selegelline; muscarinic receptor antagonists such as trihexyphenidyl, diphenylhydramine HCl; or Amantadine.

For treatment, amelioration and/or prevention of TSE, compositions can comprise a pharmaceutically acceptable dosage form comprising a compound of formula I alone or in combination with an additional active agent useful for preventing, ameliorating or reversing TSE, or for treating the symptoms associated with TSE.

For treatment, amelioration and/or prevention of CJD, compositions can comprise a pharmaceutically acceptable dosage form comprising a compound of formula I alone or in combination with an additional active agent useful for preventing, ameliorating or reversing CJD, or for treating the symptoms associated with CJD.

For treatment, amelioration and/or prevention of amyotrophic lateral sclerosis (ALS), compositions can comprise a pharmaceutically acceptable dosage form comprising a compound of formula I alone or in combination with an additional active agent useful for preventing, ameliorating or reversing ALS, or for treating the symptoms associated with ALS. Additional active agents include but are not limited to riluzol, baclofen, tizanidine, clonazepam, and dantroline.

For treatment, amelioration and/or prevention of multiple sclerosis, compositions can comprise a pharmaceutically acceptable dosage form comprising a compound of formula I alone or in combination with an additional active agent useful for preventing, ameliorating or reversing MS, or for treating the symptoms associated with MS.

For treatment, amelioration and/or prevention of Huntington's disease, compositions can comprise a pharmaceutically acceptable dosage form comprising a compound of formula I alone or in combination with an additional active agent useful for preventing, ameliorating or reversing Huntington's disease, or for treating the symptoms associated with Huntington's disease.

For treatment, amelioration and/or prevention of systemic amyloidosis, compositions can comprise a pharmaceutically acceptable dosage form comprising a compound of formula I and an additional active agent useful for preventing, ameliorating or reversing systemic amyloidosis, or for treating the symptoms associated with systemic amyloidosis. Additional active agents include but are not limited to colchicine.

For treatment, amelioration and/or prevention of primary amyloidosis, compositions can comprise a pharmaceutically acceptable dosage form comprising a compound of formula I alone or in combination with an additional active agent useful for preventing, ameliorating or reversing systemic amyloidosis, or for treating the symptoms associated with systemic amyloidosis. Additional active agents include but are not limited to colchicine.

For treatment, amelioration and/or prevention of diffuse Lewy body disease, compositions can comprise a pharmaceutically acceptable dosage form comprising a compound of formula I alone or in combination with an additional active agent useful for preventing, ameliorating or reversing Lewy body disease, or for treating the symptoms associated with Lewy body disease.

For treatment, amelioration and/or prevention of Pick's disease, compositions can comprise a pharmaceutically acceptable dosage form comprising a compound of formula I alone or in combination with an additional active agent useful for preventing, ameliorating or reversing Pick's disease, or for treating the symptoms associated with Pick's disease.

For treatment, amelioration and/or prevention of fronto-temporal dementia, compositions can comprise a pharmaceutically acceptable dosage form comprising a compound of formula I alone or in combination with an additional active agent useful for preventing, ameliorating or reversing fronto-temporal dementia, or for treating the symptoms associated with fronto-temporal dementia.

For treatment, amelioration and/or prevention of AIDS dementia, compositions can comprise a pharmaceutically acceptable dosage form comprising a compound of formula I alone or in combination with an additional active agent useful for preventing, ameliorating or reversing AIDS dementia, or for treating the symptoms associated with AIDS dementia.

Additional indications could include, but are not limited to, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, lysozyme-associated hereditary systemic amyloidosis, autism, schizophrenia, bipolar disorders, progressive supranuclear palsy, systemic lupus erythematosus, rheumatoid arthritis, spinocerebellar ataxias, Crohn's disease, ulcerative colitis, and polyneuropathy. Compositions can comprise a pharmaceutically acceptable dosage form comprising a compound of formula I alone or in combination with an additional active agent useful for preventing, ameliorating or reversing each of these conditions.

V. Methods of Treatment

The methods and compositions disclosed herein are useful in the treatment of an animal suffering from a disease or condition resulting from or associated with a pathogenic protein. The animal is not particularly limited, and can be a farm animal, for example, a cow, pig, sheep, chicken or goat, and the like, a companion animal, for example, a dog, cat, parrot, and the like, a zoo animal, for example, an elephant, or tiger, and the like, a laboratory animal, for example, a rat, mouse, transgenic mouse, and the like, a wild animal for example, a bird, fish, reptile, and the like), or a game animal, for example, elk, deer, pheasant, and the like. In preferred embodiments, the animal is a mammal, including a human patient.

In one aspect, pharmaceutical compositions comprising compounds of formula I, II, III, IV and/or V, with or without additional active agent, can be administered to a patient over a significant period of time when the patient is infected with a prion related disease such as CJD. This treatment is a non-prophylactic treatment, but rather a direct treatment of the patient, such as a human, in an attempt to cure what is now a 100% fatal disease. Compositions comprising compounds such as tafenoquine, mefloquin and compounds disclosed in Table 1 are especially preferred.

In an additional aspect, there is provided a method for reducing or retarding degeneration in a cell population of a tissue or organ subject to damage in an amyloid disease, comprising exposing the cell population to a therapeutically effective amount of a compound of formula I. In an additional embodiment, there is provided a method for reducing necrosis or apoptosis in a cell population of a tissue or organ subject to necrotic or apoptotic damage in an amyloid disease, comprising exposing the cell population to a therapeutically effective amount of a compound of formula I. The cell population can comprise any cell exhibiting damage, necrosis or apoptosis associated with an amyloid disease, and typically includes pancreatic islet cells, neurons, glia, endothelial cells, or endocrine cells.

In an additional embodiment, there is provided a method of blocking amyloid toxicity in cells, said method comprising contacting said cells with an amount of at least one compound of formula I effective to inhibit formation of amyloid oligomers and polymers. Preferably, the amyloid toxicity is selected from amyloid beta peptide toxicity, amyloid prion protein toxicity, human amylin toxicity, amyloid A protein toxicity, transthyretin toxicity, or AL amyloid toxicity.

In yet another aspect, a method is provided for reducing or retarding neurodegeneration in a cell population comprising neurons which have been exposed to an amount of a stimulus sufficient to produce at least partially protease resistant proteins resulting in neurodegeneration, the method comprising: exposing the cell population to a therapeutically effective amount of a compound of formula I. Similarly, methods are provided for inhibition of neuronal cell death in a cell population that is exposed to a protease resistant prion protein stimulus, the method comprising: administering a therapeutically effective amount of a compound of formula I to the cell population.

A. Diseases and Associated Pathogenic Proteins

Any disease that is etiologically linked to the formation and/or deposition of amyloid is contemplated for treatment according to the present invention. Amyloid diseases or disorders are associated with the formation and/or deposition of amyloid, and include, but are not limited to, Creutzfeldt-Jakob disease, scrapie, transmissible spongiform encephalopathy (TSE), Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy, AIDS dementia, systemic senile amyloidosis, prion disease, Gerstmann-Straussler-Scheinker syndrome, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, or lysozyme-associated hereditary systemic amyloidosis, and the like. The compounds of formula I can be used in a method of treatment of these amyloid diseases by administering a therapeutically effective amount of a compound of formula I and optionally a pharmaceutically acceptable carrier or diluent, and/or an additional active agent.

Methods of treating pathogenic protein diseases, like protease resistant prion related diseases, are also provided. Diseases that may be treated by the claimed methods include, but are not limited to TSE, CJD, Kuru, Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy and AIDS dementia.

Certain examples provided herein relate to the use of tafenoquine in the reduction and/or clearance of PrP^{Sc} from cells. Accordingly, in certain embodiments, the compositions and methods as described herein can be applied to obtain the reduction and/or clearance of the formation of pathogenic proteins for any protein which assumes two different conformational shapes, one of which is associated with the disease. The following is a non-limiting list of diseases with associated insoluble proteins which assume two or more different conformations.

| Disease | Pathogenic Proteins |
|---|---|
| Alzheimer's Disease | APP, Aβ peptide, α1- |
| | antichymotrypsin, tau, |
| | non-Aβ component |
| Prion diseases, Creutzfeld Jakob | Prions (PrP^{X}) including but |
| disease, scrapie and Bovine | not limited to hPrP and PrP^{Sc} |
| Spongiform Encephalopathy | |
| ALS | SOD and neurofilament |
| Pick's disease | Pick body |
| Parkinson's disease | Lewy body |
| Diabetes Type 1 | Amylin |
| Multiple myeloma-plasma cell dyscrasias | IgGL-chain |
| Familial amyloidotic polyneuropathy | Transthyretin |
| Medullary carcinoma of thyroid | Procalcitonin |
| Chronic renal failure | β₂-microglobulin |
| Congestive heart failure | Atrial natriuretic factor |
| Senile cardiac and systemic myloidosis | Transthyretin |
| Chronic inflammation | Serum amyloid A |
| Atherosclerosis | ApoA1 |
| Familial amyloidosis | Gelsolin |
| Huntington's Disease | Huntington protein (mHtt) |

Thus, a “pathogenic protein” is selected from amyloid proteins such as, but not limited to, APP, Aβ peptide, α1-antichymotrypsin, tau, non-Aβ component, PrP^{Sc}, SOD, neurofilament, Pick body, Lewy body, amylin, IgGL-chain, transthyretin, procalcitonin, β₂-microglobulin, atrial natriuretic factor, serum amyloid A, apoA1, gelsolin or Huntington protein.

It should be noted that the proteins listed above each include a number of variants or mutations which result in different strains which are all encompassed by the present disclosure. Known pathogenic mutations and polymorphisms in the PrP gene related to prion diseases are given below and the sequences of human, sheep and bovine are given in U.S. Pat. No. 5,565,186, issued Oct. 15, 1996.

MUTATION TABLE

| Pathogenic Human | | | |
|---|---|---|---|
| Sheep Bovine | Human | Sheep | Bovine |
| human mutations | Polymorphisms | Polymorphisms | Polymorphisms |
| 2 octarepeat insert | Codon 120 Met/Val | Codon 171 Arg/Glu | 5 or 6 octarepeats |
| 4 octarepeat insert | Codon 219 Glu/Lys | Codon 136 Ala/Val | |
| 5 octarepeat insert | | | |
| 6 octarepeat insert | | | |
| 7 octarepeat insert | | | |
| 8 octarepeat insert | | | |
| 9 octarepeat insert | | | |
| Codon 102 Pro-Leu | | | |
| Codon 105 Pro-Leu | | | |
| Codon 117 Ala-Val | | | |
| Codon 145 Stop | | | |
| Codon 178 Asp-Asn | | | |
| Codon 180 Val-Ile | | | |
| Codon 198 Phe-Ser | | | |
| Codon 200 Glu-Lys | | | |
| Codon 210 Val-Ile | | | |
| Codon 217 Asn-Arg | | | |
| Codon 232 Met-Ala | | | |

It should also be noted that such proteins have two different 3-dimensional conformations with the same amino acid sequence. One conformation is associated with disease characteristics and is generally insoluble whereas the other conformation is not associated with disease characteristics and is soluble. The methodology described herein is not limited to the diseases, proteins and strains listed.

B. Prophylactic Uses

As indicated here, the term “treating” means preventing, inhibiting or relieving the disease or symptom thereof. However, there are specific situations wherein a clear prophylactic treatment is indicated, i.e., the aspect of treating that involves preventing the disease. Specifically, there is an identifiable portion of the population that has inherited diseases related to prions such as CJD. Family members who have or might have an inherited trait for such disease can take a composition as described herein in order to prevent the development of the disease and/or symptoms. A second class of individuals are those who have been exposed to prions by ingesting infected food such as beef products, which were derived from cattle with BSE, and includes individuals who have spent significant amounts of time in Europe and/or other areas where the cattle were likely to have been infected with BSE. A third group of people are people who have been treated with human growth hormone derived from a human cadaver. Such individuals are at risk for the development of such diseases and would be treated with a composition as described herein. A fourth group of individuals are those that have been subjected to surgery and exposed their nerve tissue to surgical instruments, which may have been already infected with prions. Further, the individual may be an individual who has had a dura mater transplant from an individual who may have been infected with a disease such as CJD.

In treating patients who do not at the present have an amyloid disease such as a prion disease or Alzheimer's disease, but who are believed to be at substantial risk for developing such diseases, the physician should start treatment when the patient first experiences early symptoms, such as memory or cognitive problems, in Alzheimer's disease, or high blood sugar in diabetes type II, for example. In addition, there are some patients who may be diagnosed with an amyloid disease through the detection of a biological or genetic marker predictive for the amyloid disease. For example, patients having the genetic marker for APOE4 are at risk for developing Alzheimer's disease. In these situations, even though the patient does not have symptoms of the disease, the administration of pharmaceutical compositions comprising compounds of formula I, with or without additional active agent, may be started before symptoms appear and treatment can be continued indefinitely to prevent or delay the outset of the disease.

VI. Modes of Administration and Dosages

The compositions of the invention may be administered to the patient by a variety of different means. The means of administration will vary depending upon the intended application. As one skilled in the art would recognize, administration of the compositions can be carried out in various fashions, for example, via topical administration, including, but not limited to, dermal, ocular and rectal; transdermal, via passive or active means, e.g., using a patch, a carrier, or iontophoresis; transmucosal, e.g., sublingual, buccal, rectal, vaginal, or transurethral; oral, e.g., gastric or duodenal; parenteral injection into body cavity or vessel, e.g., intraperitoneal, intravenous, intralymphatic, intratumoral, intramuscular, interstitial, intraarterial, subcutaneous, intralesional, intraocular, intrasynovial, intraarticular; or via inhalation, e.g., pulmonary or nasal inhalation, using e.g., a nebulizer. Oral administration is preferred in certain patient classes, while for others, rectal or parenteral administration may be preferred. Oral administration can utilize immediate release or controlled release compositions. The mode of administration will generally affect dosing in that some modes are more efficient at delivery of active compound to the desired site than others.

Direct infusion methods, and use of osmotic pumps, which administer a composition as disclosed herein directly to cells to be treated (e.g., brain tissue), can also be used.

Any suitable dosage can be given in the methods described herein. Compounds of formula I and the carrier and the amount will vary widely depending on the species of the animal, body weight, and disease being treated. The dosage administered will, of course, vary depending upon known factors, such as the pharmacodynamic characteristics of compounds of formula I and their mode and route of administration; the age, health and/or weight of the recipient; the nature and extent of the symptoms; the kind of concurrent treatment; the frequency of treatment; and the effect desired, for example. Those skilled in the art will adjust dosing as needed, beginning with smaller doses and increasing gradually while monitoring side effects and the effect of the drug on the disease being treated. With an oral composition of tafenoquine or mefloquin, for example, dosing can be generally in an amount of about 0.5 mg to about 10,000 mg/day/75 kg of body weight of the animal being treated. Thus, a human dose can be about 0.5-10,000 mg/day, and larger animals are given larger doses in proportion to their weight. Typically, the therapeutic amount is in the range of from about 0.1 to about 1000 mg/day, or in the range of from about 15 to about 1500 mg/day, or in the range of from about 1 to about 100 mg/day, or in the range of from about 5 to about 50 mg/day.

Dosages of agents coadministered with compounds of formula I are selected based on usual dosages known for these agents and adjusted as necessary based on individual patient health and needs, drug-drug interactions, and other contraindications.

A. Pharmaceutical Compositions and Kits

The present disclosure further concerns pharmaceutical compositions incorporating compounds of formula I. Such pharmaceutical compositions should contain a therapeutic or prophylactic amount of compounds of formula I. The pharmaceutically acceptable carrier can be any compatible, non-toxic substance suitable to deliver the compounds to an intended host. Pharmaceutically acceptable adjuvants, buffering agents, dispersing agents, and the like may also be incorporated into the pharmaceutical compositions. Preparation of pharmaceutical conditions is well described in the medical and scientific literature. See, for example, Remington's Pharmaceutical Sciences, Mack Publishing Company, Easton, Pa., 16th Ed., 1982.

The therapeutic compounds may be administered alone or in combination with pharmaceutically acceptable carriers or diluents by any of the routes previously indicated, and such administration may be carried out in single or multiple doses. More particularly, the therapeutic compounds used in the methods of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Such carriers include solid diluents or fillers, sterile aqueous media and various non-toxic organic solvents, for example.

Capsule or tablets can be easily formulated and can be made easy to swallow or chew; other solid forms include granules, and bulk powders. Tablets can contain suitable binders, lubricants, diluents, disintegrating agents, coloring agents, flavoring agents, flow-inducing agents, and melting agents. For oral administration, tablets containing various excipients such as, but not limited to, microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For oral administration in liquid dosage form, compounds of formula I can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Oral dosage forms optionally contain sweeteners, flavorants and coloring agents. Parenteral and intravenous forms can also include minerals and other materials to make them compatible with the type of injection or delivery system chosen. Thus, the present disclosure provides compositions for administration to a host, where the compositions comprise a pharmaceutically acceptable solution of a compound of formula I in an acceptable carrier, as described above.

Gelatin capsules can contain compounds of formula I and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Lipophilic solvents or carriers, such as DMZ, an organic solvent, and lipids such as phosphatidyl choline or cholesterol can be utilized. Solutions for parenteral administration preferably contain a water soluble salt of a compound of formula I or a liposome, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules

For parenteral administration, solutions of a therapeutic compound used in the methods of the present invention can be prepared in substances selected from, but not limited to, oils, such as sesame or peanut oil, or aqueous propylene glycol, or any other pharmaceutically acceptable formulation may be employed. The aqueous solutions should be suitably buffered if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Compounds of formula I can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of formula I can also be coupled with soluble polymers as targetable drug carriers. Such polymers can include polyvinylpyrrolidone, pyran copolymer, polyhydroxylpropylmethacrylamidephenol, polyhydroxyethylaspartamidephenol, or polyethyleneoxide-polylysine substituted with palmitoyl residues. Furthermore, compounds of formula I can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The pharmaceutical compositions just described are suitable for systemic administration to the host, including parenteral, topical, and oral administration, as well as intracranial administration.

A unit dosage can comprise compounds of formula I in a mixture with other compounds or other inhibiting compounds. The dosage unit can also comprise diluents, extenders, carriers and the like. The unit can be in solid or gel form such as pills, tablets, capsules and the like or in liquid form suitable for oral, rectal, topical, intravenous injection or parenteral administration or injection.

Compounds of formula I can be mixed with a pharmaceutically acceptable carrier. This carrier can be a solid or liquid and the type is generally chosen based on the type of administration being used. compounds of formula I can be co-administered with additional pharmaceutically active agents in the form of a tablet or capsule, as an agglomerated powder or in a liquid form. Examples of suitable solid carriers include lactose, sucrose, gelatin and agar.

Compositions containing compounds of formula I can be administered for prophylactic and/or therapeutic treatments of neurodegenerative disease. In therapeutic application, compositions are administered to a patient already affected by the particular neurodegenerative disease in an amount sufficient to cure or at least partially arrest the condition and its complications. An amount adequate to accomplish this is defined as a “therapeutically effective dose” or “efficacious dose.”

Compounds of formula I can be an effective pharmaceutical for therapy in pathogenic protein diseases, like prion related diseases in mammals. Compounds of formula I is known to cross the blood-brain barrier and PrP^{Sc} inhibiting concentrations of compounds of formula I can be achieved in the brain by daily dosages.

In methods as disclosed herein, compounds of formula I can form the active ingredient, and are typically administered in admixture with suitable pharmaceutical diluents, excipients, or carriers (collectively referred to herein as a pharmaceutically acceptable carrier or carrier materials) suitably selected with respect to the intended form of administration, that is, oral tablets, capsules, elixirs, syrups and the like, and consistent with conventional pharmaceutical practices. For instance, for oral administration in the dosage unit form of a tablet or capsule, compounds of formula I can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like.

Compounds of formula I are able to cross the blood-brain barrier of a human to produce a therapeutically efficacious concentration in cerebrospinal fluid and CNS tissues (e.g., cortical neurons). Other approaches to enhancing delivery of compounds of formula I, particularly across the blood-brain barrier, can utilize osmotic pumps and/or pharmacologic-based procedures involving drug latentiation or the conversion of hydrophilic drugs into lipid-soluble drug.

In some embodiments, a physiologically compatible salts of compounds of formula I can be used in the methods described herein. Non-limiting examples of such salts include organic and inorganic acids, as exemplified by hydrochloric acid, sulfuric acid, phosphoric acid, formic acid, acetic acid, oxalic acid, tartaric acid, maleic acid, benzoic acid, succinic acid, fumaric acid, levulinic acid, salicyclic acid, citric acid, isocitric acid, adipic acid, lactic acid, α-ketoglutaric acid, malic acid, malonic acid, glyceric acid, mevalonic acid, glucuronic acid, neuraminic acid, glutaric acid, aspartic acid, gluconic acid, mandelic acid, ascorbic acid, lactobionic acid, glucoheptonic acid, glutamic acid, nicotinic acid, pantothenic acid, folic acid, adenylic acid, geranylic acid, cytidylic acid and inosic acid.

A composition suitable for use in some embodiments of the present methods comprises tafenoquine succinate as manufactured by GlaxoSmithKline Inc. (Research Triangle Park, NC).

The present disclosure also provide pharmaceutical kits useful, for example, for the treatment of pathogenic protein diseases, which comprise one or more containers containing a pharmaceutical composition comprising a therapeutically effective amount of a compound of formula I, with or without additional active agents. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit. In the present disclosure it should be understood that the specified materials and conditions are important in practicing the methods disclosed herein but that unspecified materials and conditions are not excluded so long as they do not prevent the benefits of the disclosed methods from being realized.

The present disclosure also provides the use of compounds of formula I to slow, arrest, or reverse the development of a degenerative diseases, including a neurodegenerative disease, in a human patient; an efficacious amount of compounds of formula I with or without additional active agent is administered to the patient to inhibit progression of the disease.

B. Livestock Feed

Some aspects of the present disclosure concern combinations of compounds of formula I combined with a livestock feed. Compounds of formula I can be combined with a livestock feed derived from an animal source, such as meat or bone meal and more particularly animal material that includes ground material from the central nervous system from another animal. Such feed may be infected with prions. However, compounds of formula I can be combined with plant derived materials used in livestock feed in order to prevent or treat prion infections in the animal eating the feed. More particularly, compounds of formula I can be added to animal feed or feed stuffs used to feed any type of animal and particularly for livestock. The feed stuff compositions disclosed herein are intended to provide nutritional requirements of a variety of animals, including cattle, poultry, swine, sheep, goats, other monogastric or ruminant livestock. The compositions generally vary according to the type of animals to which the feedstuff will be given. Examples of various animal feedstuff components can be found in U.S. Pat. No. 6,207,217, U.S. Pat. No. 6,203,843, U.S. Pat. No. 5,786,007, U.S. Pat. No. 4,225,621, U.S. Pat. No. 4,161,543 and U.S. Pat. No. 4,062,988.

Generally, when the term “feedstuff” is used with respect to the present disclosure, the term comprises all types of plant and animal components. In some embodiments, “feedstuff” includes organic components such as proteins, crude fiber, acid detergent fiber, neutral detergent fiber, vitamins and minerals. Typical compositions of feedstuffs for livestock include, but are not limited to, the following components: alfalfas, ammonium sulfate, barleys, beet pulps, blood meal, bluestem grass, brewers grains and yeast, brome grass, calcium carbonate, canary grass, carrot pulp, roots and tops, cattle manure, cheatgrass, clovers, coffee grounds, corn and corn plants, cottonseed, defluorinated phosphate, diammonium phosphate, dicalcium phosphate, distillers grain barley, distillers grain corn, feathermeal hydrolyzed, garbage (municipal), grain screenings and grain dust, grape pomace, grass silage, hominy feed, hop leaves, vines and spent hops, limestone, linseed meal, all types of hay including meadow hay, meat and bone meal (MBM), milo grain, mint slug silage, molasses beet, cane, citrus and wood, monoammonium phosphate, mono-dicalcium phosphate, navy beans, all types of oats including oat hay, oat silage, oat straw, oat grain, groats, oat meal, oat mill byproducts and oat hull, orange pulp, orchard grass, pea vines, peanut hulls, skins and meal, potato vine, potatoes and potato waste, poultry fat and poultry litter and manure, prairie hay, rapemeal solvent, rye straw and grain, safflower meal, sagebrush, sorghum stover and silage, soybeans and soybean hull, sudangrass hay and silage, sunflower meal and hulls, timothy hay and silage, tomatoes, triticale silage, urea, wheat bran, wheat grass, wheat grain, wheat shorts and wheat straw. Further, the above feedstuff components are set forth above serve merely as examples and are not intended to be comprehensive or limiting. As such, suitable feedstuffs for the present disclosure may comprise additional components not provided in the list above.

Of the above listed feed components, meat and bone meal (MBM) stands out as one the richest sources of energy and minerals. Typically, the crude protein content of MBM is about 50%. See, Hamilton, C. R., “Meat and Bone Meal,” Esteem Products. Vol. 1 (1). MBM is thus one of the most efficient feed components. MBM is produced as a by-product from the removal of fat from animal tissues through rendering. The rendering process produces a finely ground, dry residue of animal by-products pressure cooked and stabilized by high temperature steam in closed tanks. The fat can be skimmed off and the solid residue is pressed to remove as much of the fat and water as possible. As defined and regulated by the Association of American Feed Control Officials (AAFCO), MBM is the rendered product from mammal tissues, including bone, exclusive of any added blood, hair, hoof, horn, hide trimmings, manure, stomach and rumen contents, except in such amounts as may occur unavoidably in good processing practices. As such, neuronal tissues are included in MBM products. See, “The BSE Inquiry” § 9.15 at www.bse.org.uk/rep-ort/volume7/chapteh2.htm.

The present disclosure encompasses feedstuff as defined, in combination with compounds of formula I. In some embodiments, a compounds of formula I is added to feedstuff and fed to an animal and in particular to domesticated livestock farm animals such as cows, pigs, sheep, goats, horses, chickens, etc. Compounds of formula I can be added in an amount sufficient to “treat” the animal. The amount will vary based on factors such as the type of animal and its size. In some embodiments, dosing is such that the animal will receive about 0.1 mg to about 10,000 mg/day/kg of weight of the animal.

VII. Methods of Determining Efficacy of Compounds Against Amyloid Formation

One skilled in the art is able to practice the screening procedures required to determine whether or not a particular compound of formula I possesses activity against pathogenic proteins. Example 1 describes how to ascertain the inhibition of PrP^{Sc} formation in scrapie-infected mouse neuroblastoma cells, either RML (Bosque et al. (2000) J. Virol. 74, 4377-4386) or 22L. Briefly, test compounds are added to scrapie-infected mouse neuroblastoma cells and the cells are incubated for 5 days at 37° C. in a CO₂ incubator before being lysed and the amount of PrP^{Sc} formation determined.

Yeast prion (PSI⁺) transmission is mediated by the self propagating aggregation of the translation termination factor Sup35, which is comprised of a prion-determining region (NM) fused to a translational termination region, that results in a nonsense suppression phenotype (Uptain, et al. (2001) EMBO J. 20, 6236-6245). Polymerization of Sup35 is mediated by the yeast Sup-NM protein, which consists of glutamine/asparagine-rich N-terminal domain (N) and a charged middle domain (M) (Bradley et al. (2004) Mol. Microbiol. 51, 1649-1659, forming self replicating amyloid fibers (Glover et al. (1997) Cell 89, 811-819; King et al. (1997) Proc. Natl. Acad. Sci. U.S.A. 94, 6618-6622). When introduced into yeast these fibers initiate the (PSI⁺) prion state (Sparrer et al. (2000) Science 289, 595-599; King et al. (2004) Nature 428, 319-323; Tanaka et al. (2004) Nature 428, 323-328), showing that the amyloids are in fact the infectious yeast prion element underlying (PSI⁺). Sup-NM amyloid formation in vitro reflects closely the induction and propagation of Sup35^{PSI+} in vivo, and PSI⁺ variants resemble the mammalian prion strains in that they can propagate in the same host strain with different disease latencies and pathological manifestations (Uptain et al., 2001).

As the yeast prion state (PSI⁺) is an excellent model for delineating amyloid fiber formation and prion propagation (Collins et al. (2004) PLoS Biol. 2, e321), polymerization assays of the yeast prion Sup-NM can be performed to demonstrate activity of agents against yeast prion formation and propagation. Example 4 demonstrates the use of the yeast prion system for determining inhibition of prion polymerization and PSI⁺ induction. Prions are self-propagating in nature and amyloid formation is common in most, but not all TSEs. Therefore, the yeast prion model is likely applicable to prion formation and propagation in other systems, including TSEs affecting mammals and birds.

In addition, the inhibition of the oligomerization of amyloid peptide (inhibition of amyloid fibrillogenesis) can be estimated by the method described by Klunk, et al. using Congo Red (Klunk, W. E., Jacob, R. F., Mason, R. P. (1999) “Quantifying amyloid β-peptide aggregation using the congo red-Aβ spectrophotometric assay” Anal. Biochem. 266, 66-76). This assay method is described in Example 7, and was used to demonstrate inhibition of fibrillogenesis by compounds of formula I. Inhibition of amyloid fibrillogenesis and disassociation of fibrils can also be demonstrated using the gel assay described in Example 8.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, polymer chemistry, biochemistry and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

### EXAMPLES

The following examples are put forth so as to provide those of ordinary skill in the art with a complete disclosure and description of how to make and use the presently disclosed compositions and methods, and are not intended to limit the scope of what the Applicants regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All solvents were purchased as HPLC grade, and all reactions were routinely conducted under an inert atmosphere of argon unless otherwise indicated. Unless otherwise indicated, the reagents used were obtained from the following sources: Tafenoquine succinate was provided by GlaxoSmithKline, Mefloquine monohydrochloride was supplied by Hoffmann-La Roche Inc., amyloid peptide gels, and gel stains from Invitrogen (Camarillo, Calif.), organic solvents, from Aldrich Chemical Co., Milwaukee, Wis.; gases, from Matheson, Seacaucus, N.J.

### Example 1

### The Formation and Accumulation of PrP^{Sc} in Scrapie-Infected Neuroblastoma Cells is Inhibited by Tafenoquine

Inhibition of PrP^{Sc} formation was tested in scrapie-infected mouse neuroblastoma cells as follows. Approximately 20,000 RML (Bosque et al. (2000) J. Virol. 74, 4377-4386) or 22L scrapie-infected mouse neuroblastoma cells were added to each well of a 96 well plate in 100 μL of medium prior to the addition of tafenoquine. 22L-Infected cells were developed by re-infection of RML-infected mouse neuroblastoma cells (N2a) cured by 7 passages in medium containing 1 μg/mL pentosan polysulfate (Priola et al. (1994) Infect. Agents Dis. 3, 54-58). The cured cells were re-infected by incubation with PrP^{Sc} purified from mouse brains infected with 22L strain of scrapie. The neuroblastoma cells reinfected with 22L scrapie have stably expressed PrP^{Sc} for over 70 passages. The cells were allowed to settle for 4 hours before tafenoquine was added.

10 mM solutions of tafenoquine were prepared and diluted in DMSO and then into PBS prior to being introduced to the cell medium. 5 μL of solutions were added to the cell medium. DMSO concentrations in the cell media were never higher than 0.5% (v/v). After tafenoquine was added, the cells were incubated for 5 days at 37° C. in a CO₂ incubator before being lysed. Prior to cell lysis, the cells were inspected by light microscopy for toxicity, bacterial contamination, and density compared to controls. After removal of the cell media, 50 μL of lysis buffer was added to each well. Lysis buffer was composed of 0.5% (w/v) Triton X-100, 0.5% (w/v) sodium deoxycholate, 5 mM Tris-HCl, pH 7.4 at 4° C., 5 mM EDTA, and 150 mM NaCl. Five minutes after adding lysis buffer, 25 μL of 0.1 mg/mL PK (Calbiochem) in TBS was added to each well and incubated at 37° C. for 50 minutes. 225 μL of 1 mM Pefabloc (Boehringer Mannheim) was then added to each well to inhibit PK activity. 250 μL of 1 mM Pefabloc was added to samples that were not PK-treated.

As shown in FIG. 2, tafenoquine was able to inhibit PrP^{Sc} formation in the 22L- and RML-infected N2a cells after five days of growth in the presence of tafenoquine. The activity was greater against RML- than 22L-infected cells. The IC₅₀ value for tafenoquine was 332 ng/mL (0.72 μM) in the RML-infected cells, compared to an IC₅₀ of (>1000 ng/mL) (>2.2 μM) for the 22L-infected cells. Thus, both the formation and accumulation of PrP^{Sc} in mammalian cells was inhibited by the administration of tafenoquine.

### Example 2

### The Formation and Accumulation of PrP^{Sc} in Scrapie-Infected Neuroblastoma Cells is Inhibited by Mefloquin

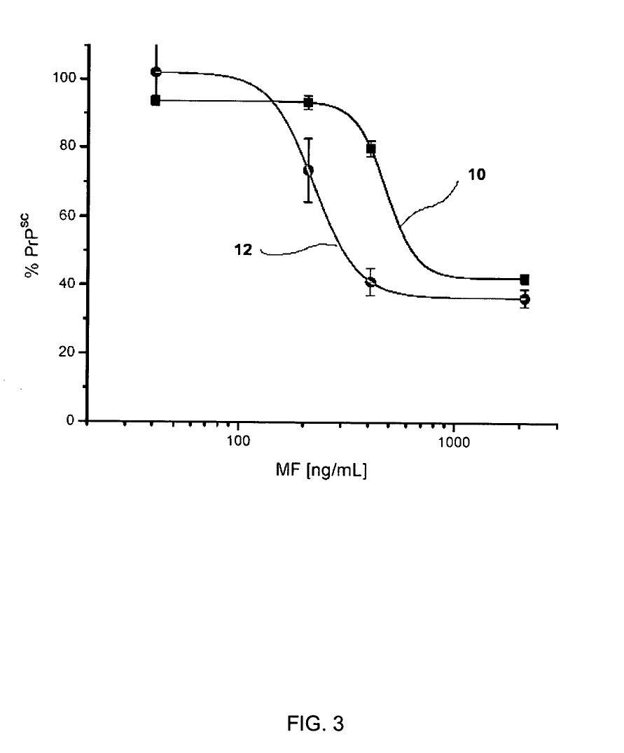

The procedure described in Example 1 was repeated, using mefloquin as the active agent. As shown in FIG. 3, mefloquine was able to inhibit PrP^{Sc} formation in the 22L- and RML-infected ScN2a cells after five days of growth in the presence of mefloquine. The activity was greater against RML- than 22L-infected cells. The IC₅₀ value for mefloquine was 224 ng/mL (0.54 μM) in the RML-infected cells, compared to an IC₅₀ of 482 ng/mL (1.2 μM) for the 22L-infected cells. Thus, both the formation and accumulation of PrP^{Sc} in mammalian cells was inhibited by the administration of mefloquin.

### Example 3

### The Formation and Accumulation of PrP^{Sc} in Scrapie-Infected Neuroblastoma Cells is Inhibited by Compound 3

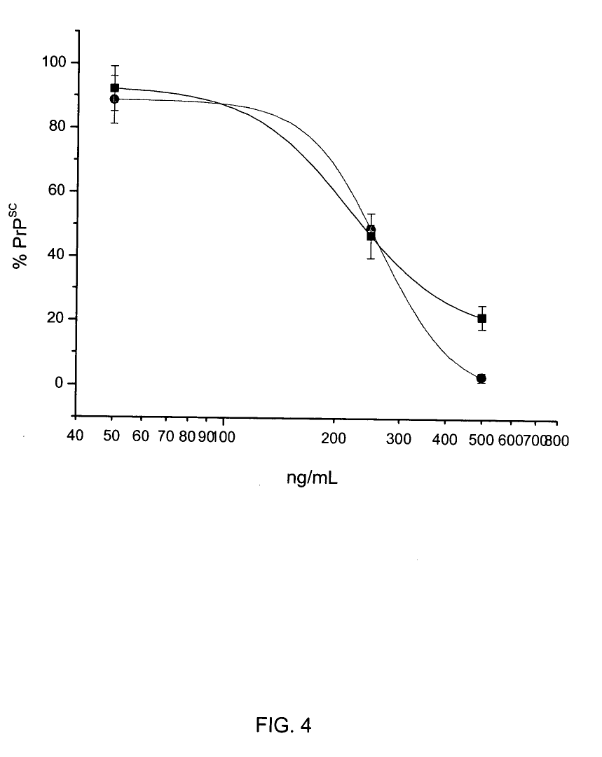

The procedure described in Example 1 was performed, using compound 3 as the active agent. As shown in FIG. 4, test compound 3 (shown in Table 1 above) was able to inhibit PrP^{Sc} formation in the 22L- and RML-infected ScN2a cells (squares and circles, respectively) after five days of growth in the presence of compound 3. The IC₅₀ value for compound 3 was 264 ng/mL (0.54 μM) in the RML-infected cells, compared to an IC₅₀ of 220 ng/mL (1.2 μM) for the 22L-infected cells. Thus, both the formation and accumulation of PrP^{Sc} in mammalian cells was inhibited by the administration of compound 3.

### Example 4

### Tafenoquine Inhibits Yeast Amyloid Polymerization

Polymerization assays of the yeast prion protein Sup-NM with or without tafenoquine were performed using thioflavin T measurements as described by Collins, et al., PLoS Biol. 2(10), e321). Concentrated stocks of NM protein stored in either 6 M GdnHCl or 4 M GdnHCl and 4 M urea were diluted at least 200-fold into buffer C (5 mM potassium phosphate and 150 mM sodium chloride [pH 7.4]). Sup-NM seed was added immediately before observation. Fluorescence was monitored in a fluorescence plate reader (Molecular Devices, Sunnyvale, Calif., USA; 442 nm excitation and 483 nm emission). Reactions were carried out at 25° C. with 3 seconds of shaking between each measurement.

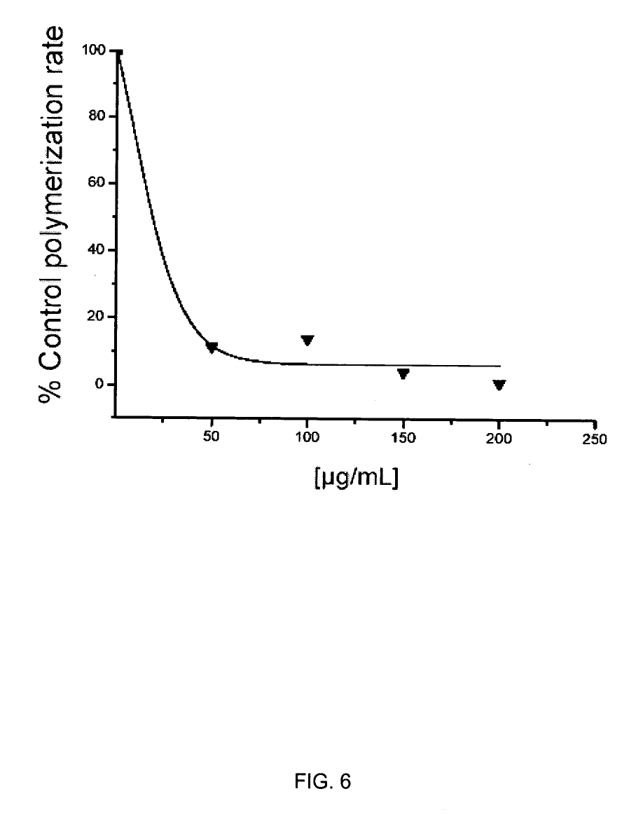

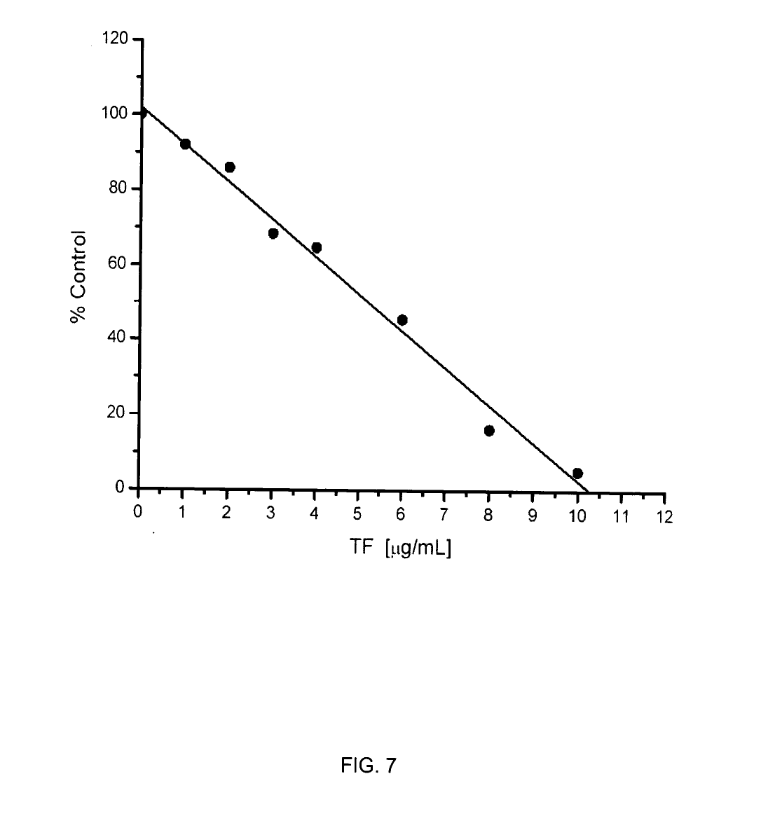

Tafenoquine (TF) was able to inhibit in a dose-dependent manner the amyloid formation of yeast prion protein, Sup-NM (FIGS. 5-7). As shown in FIG. 5, the polymerization of the yeast prion protein Sup-NM exhibits dose dependent inhibition in the presence of TF concentrations up to 200 μg/mL. The control experiment using DMSO alone is indicated by reference numeral (20), and tafenoquine inhibition is shown at the following concentrations (μg/mL): 50 (24), 100 (22), 150 (28), and 200 (26), demonstrating nearly complete inhibition of amyloid polymerization. FIG. 6 demonstrates the inhibition curve as a function of tafenoquine concentration expressed as % control polymerization rate. The IC₅₀ for tafenoquine inhibition of polymerization was determined to be 5.2±1.1 μg/mL (FIG. 7).

PSI⁺ Induction Assay in Yeast

The induction of yeast PSI⁺ (the yeast prion protein Sup35) was performed as described in published methods (Santoso et al. (2000) Cell 100, 277-288). Briefly, PSI⁻ cells containing a pRS426 Cp-NM-GFP plasmid were incubated for 24 h with 50 μM copper sulfate plus or minus drugs at 30° C. with shaking. After incubation each cell mixture was diluted 5000-fold in water, from which 100 μL was added to two different types of agar plates: (1) SD Complete (both PSI⁺ and PSI⁻ cells were able to grow on these plates; (2) SD-Ade (only PSI⁺ cells were able to grow on the plates lacking adenine (Ade)). The plates were incubated at 30° C. for 48 h or until the yeast colonies became visible. The number of colonies was next counted on all plates to determine the fraction of cells that have converted to the PSI⁺ prion state (obtained by dividing the number of colonies on the SD-Ade plate by the number of colonies on the SD Complete plate).

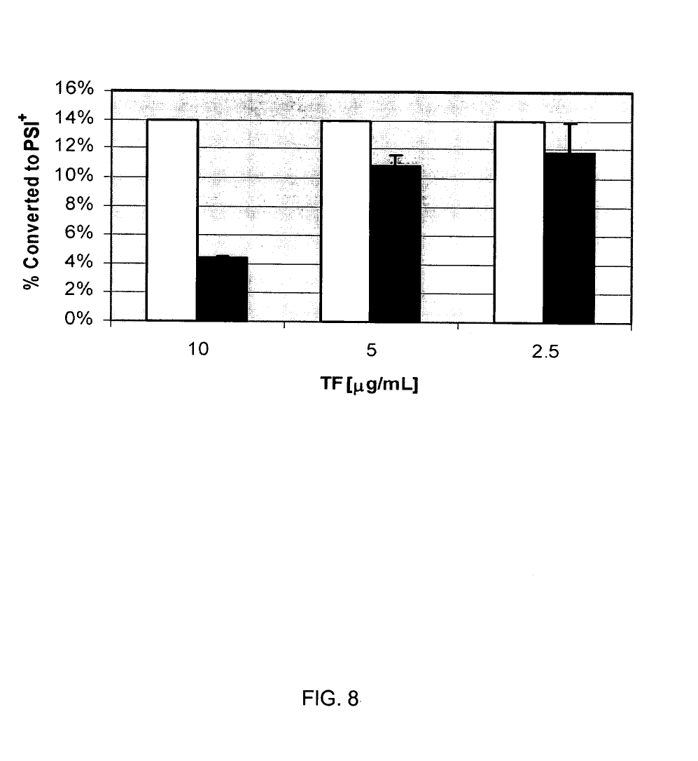

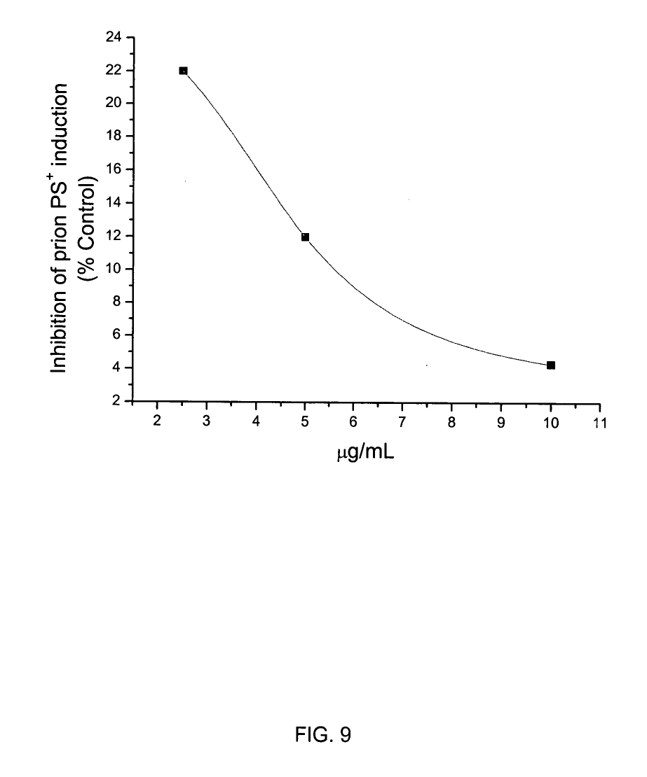

The results are shown in FIG. 8, which demonstrates the concentration dependent inhibition of PSI⁺ induction in yeast cells by DMSO (control, white bars), and tafenoquine (black bars). FIG. 9 further demonstrates that the IC₅₀ for PSI⁺ induction in the presence of tafequinone was determined to be 4.5 μg/mL.

### Example 5

### Mefloquin Inhibits Yeast Amyloid Polymerization

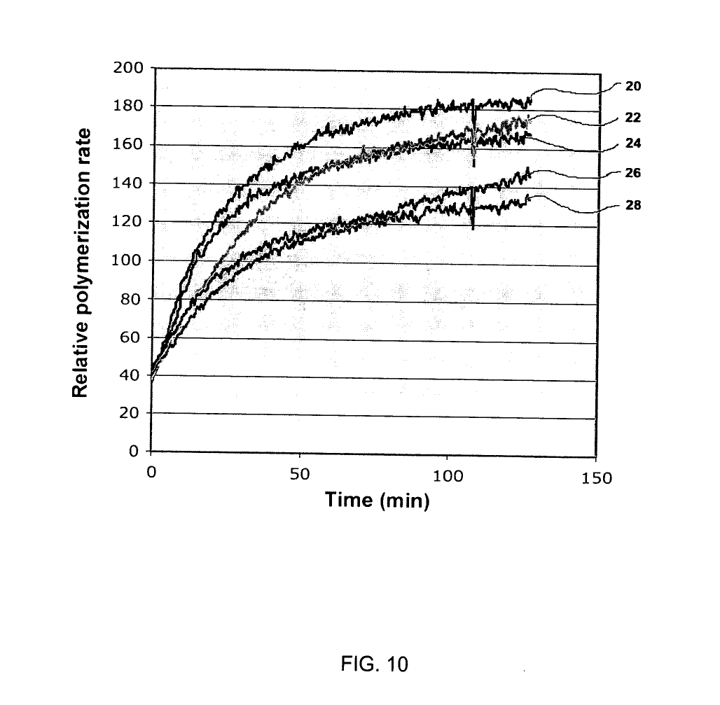

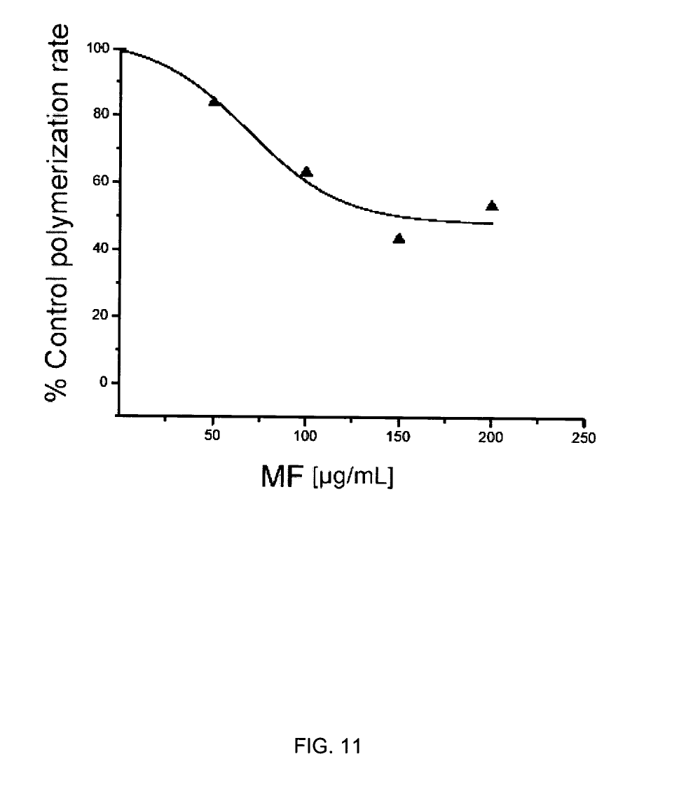

The ability of Mefloquin to inhibit the polymerization of the yeast prion protein Sup-NM was assessed as described above. Mefloquine (MF) was able to inhibit the amyloid formation of yeast prion protein, Sup-NM in a dose-dependent manner. FIG. 10 shows the results of polymerization assays of the yeast prion protein Sup-NM using thioflavin T measurements in the presence of mefloquine. At 150 μg/mL of mefloquine, there was approximately 55% inhibition of the polymerization. FIG. 11 demonstrates the concentration dependent inhibition of mefloquin on yeast prion polymerization.

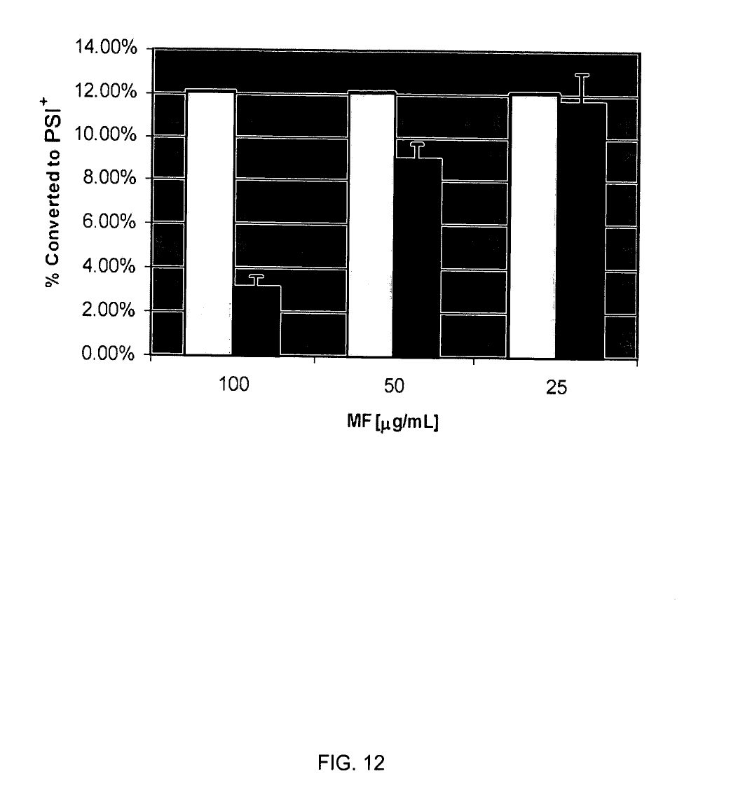

Mefloquin was also shown to inhibit the induction of yeast PSI⁺ (Sup35 protein) using the protocol described above. FIG. 12 demonstrates the concentration dependent inhibition of mefloquin on yeast prions determined by the inhibition of the induction of PSI.

### Example 6

### Compound 3 Inhibits Yeast Amyloid Polymerization

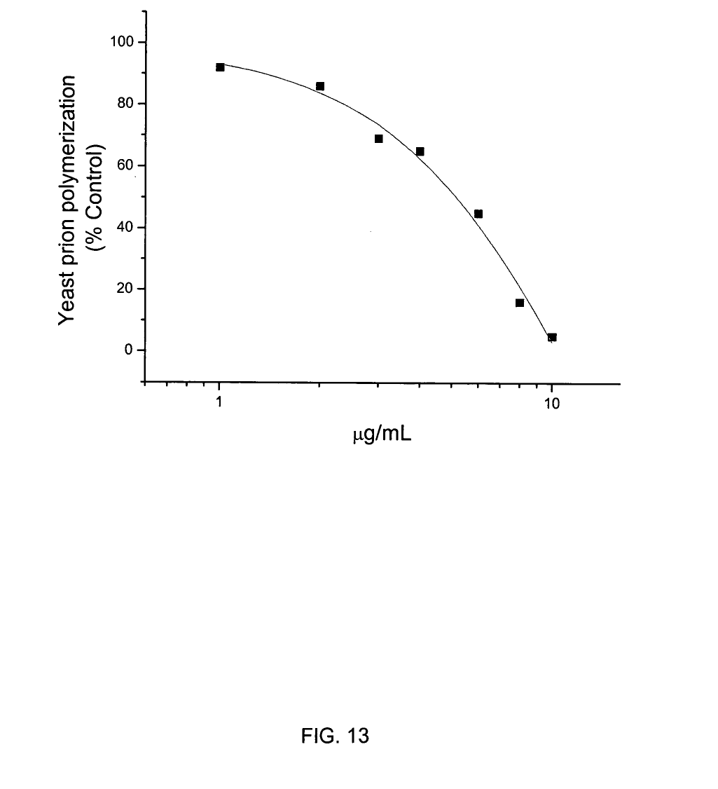

The ability of compound 3 (a compound of formula 1 shown in Table 1) to inhibit the polymerization of the yeast prion protein Sup-NM was assessed as described above in Example 4. Compound 3 was able to inhibit the amyloid formation of yeast prion protein, Sup-NM in a dose-dependent manner. FIG. 13 shows the results of polymerization assays of the yeast prion protein Sup-NM using thioflavin T measurements in the presence of compound 3. As shown in FIG. 13, compound 3 exhibits a concentration dependent inhibition on yeast prion polymerization. The IC₅₀ for compound 3 inhibition of polymerization was determined to be about 15 μg/mL.

### Example 7

### Compound 8 Exhibits an Inhibitory Effect on Amyloid Fibrillogenesis

The inhibition of the oligomerization of amyloid peptide was estimated by the method described by Klunk, et al. using Congo Red (Klunk, W. E., Jacob, R. F., Mason, R. P. (1999) “Quantifying amyloid β-peptide aggregation using the congo red-Aβ spectrophotometric assay” Anal. Biochem. 266, 66-76). Briefly, amyloid peptides were prepared in 40 μM in Tris-Acetate (40 mM:8 mM) buffer. Compounds to be tested for inhibition of fibrillogenesis were dissolved in DMSO, and then were diluted 100 fold into Tris-Acetate buffer, keeping DMSO constant at 1% (v/v). Aliquots of 100 μl of amyloid peptide were incubated with test compounds at room temperature for 5 days before measuring the absorbance. BSA was included in the buffer as a negative control. After incubation, 20 μl of 35 μM congo red was added to the 100 μl of incubated amyloid peptides to a final concentration of 7 μM. The absorbance of Congo Red at 541 nm was read using a Perkin Elmer Victor3 1420 Multilabel Counter. The inhibition of oligomerization was calculated using the following formula:
Inhibition=(Abs541 of sample−Abs541 of control)/(Abs541 of Amyloid peptide−Abs541 of BSA).

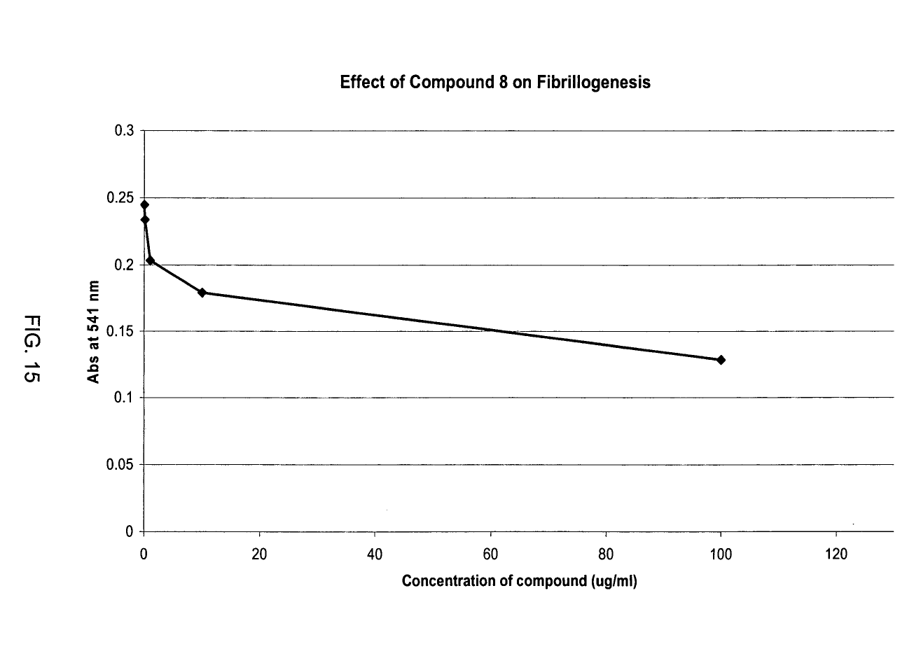

The results are shown in FIG. 14, where the absorbance is plotted as a function of wavelength for amyloid peptide incubated with different concentrations of compound 8. As shown in FIG. 14, compound 8 exhibits a concentration dependent inhibition of amyloid fibrillogenesis. The IC₅₀ for inhibition of fibrillogenesis was determined to be approximately 5 μg/mL (FIG. 15).

### Example 8

### Mefloquin Inhibits Amyloid Fibrillogenesis

Mefloquin was tested in an amyloid fibrillogenesis gel assay to demonstrate its effect on the formation and stability of amyloid fibrils.

Determination of Effect of Compounds on Amyloid Fibrilogenesis: Methods

Amyloid peptide was purchased from Biosource and 40 μM and 60 μM solutions of peptide in Tris-Acetate (40 mM:8 mM) buffer were prepared. The 40 μM solution was used in fibrillogenesis assay, as described below. The 60 μM peptide solution was undisturbed for six months and allowed to form fibrils, and was utilized in a fibril disassociation assay.

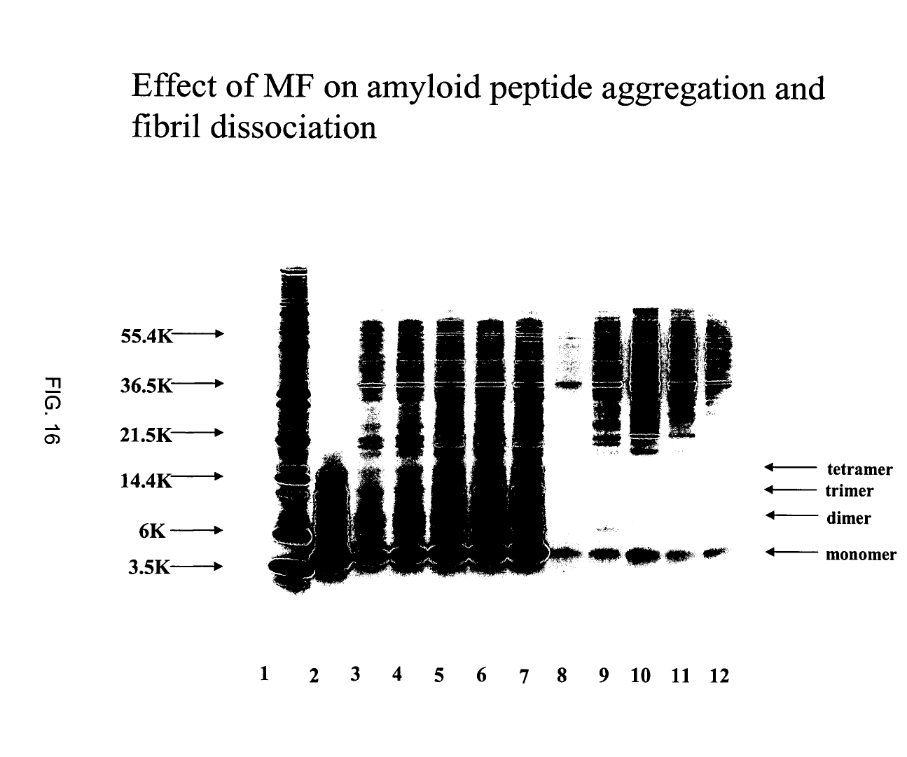

Aliquots of amyloid peptide (100 μl) were incubated with test compounds at room temperature for 5 days before performing electrophoresis. Mefloquin was dissolved in DMSO and diluted 100 fold into the final reaction mixture, keeping the DMSO concentration constant at 1% (v/v). After incubation, 10 ul of each sample was mixed with 2.5 μl of 4×SDS-sample buffer (Invitrogen), and loaded onto a precast 12% BT gel (Invitrogen). The electrophoresis was run with MES running buffer (Invitrogen) at 200V, and the gels were stained using a silverXpress silver staining kit (Invitrogen). The results are shown in FIG. 16. The lane assignments are as follows: Lane 1: molecular weight markers, 2; 40 μM fresh Aβ42, Lanes 3-7: 40 μM Aβ42 with mefloquin from 0, 3, 10, 30 to 100 μg/ml. Lanes 8-12: 10 μl Aβ42 fibrils with mefloquin from 0, 3, 10, 30 to 100 μg/ml.

As shown in FIG. 16, incubation of amyloid peptides in the absence of mefloquin (lane 3) results in the formation of high molecular weight oligomers and a reduction in the amount of monomeric peptide (fibrillogenesis), while incubation in the presence of mefloquin (lanes 3-7) results in a concentration dependent increase in the amount of monomer relative to control, as well as other low molecular weight oligomeric species, indicating that mefloquin inhibited fibrillogenesis of amyloid peptide. Disassociation of amyloid fibrils (lanes 8-12) is also shown by the increase of monomeric amyloid peptide in the presence of mefloquin.

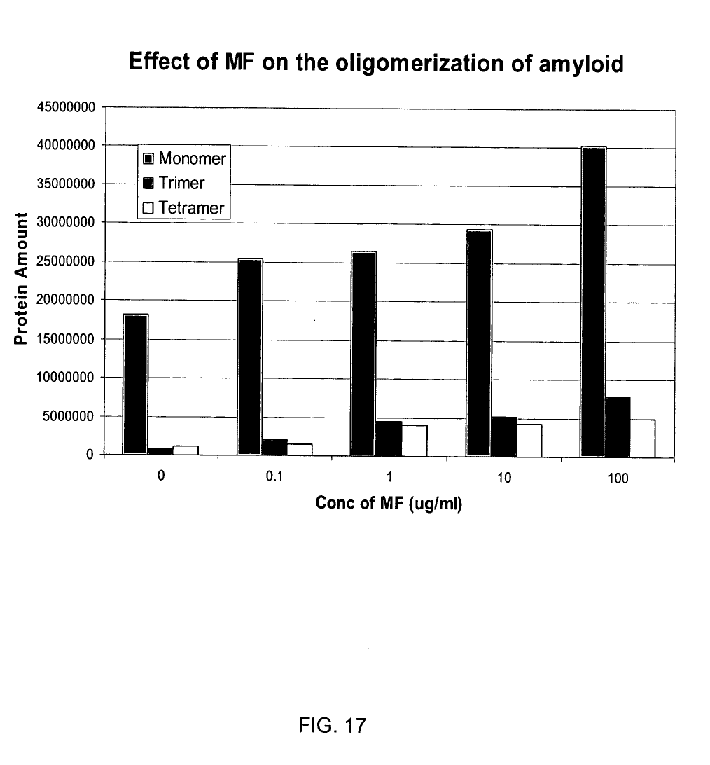

Gels were photographed and scanned, and the band densities was analyzed using NIH Imagequant, version 5.2 (GE Healthcare, Sunnyvale, Calif.). The results are shown in FIG. 17, which demonstrates that low molecular weight forms of peptide are favored in the presence of mefloquin, especially monomeric peptide, and that mefloquin inhibits the formation of amyloid fibrils.

### Example 9

### Compound 3 Inhibits Amyloid Fibrillogenesis

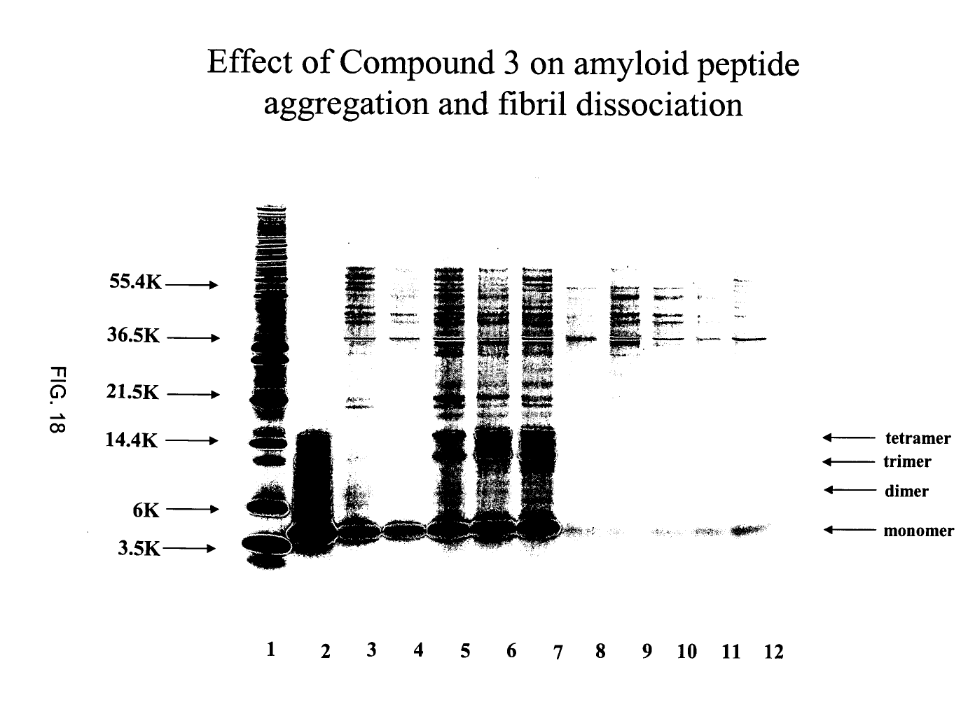

Compound 3 was tested in the amyloid fibrillogenesis and fibril disassociation gel assay described in Example 8 to demonstrate its effect on the formation and stability of amyloid fibrils. The results are shown in FIG. 18. The lane assignments are as follows: Lane 1: molecular weight markers, 2; 40 μM fresh Aβ42, Lanes 3-7: 40 μM Aβ42 with compound 3 from 0, 3, 10, 30 to 100 μg/ml. Lanes 8-12: 60 μM Aβ42 (fibrils) with compound 3 from 0, 3, 10, 30 to 100 μg/ml.

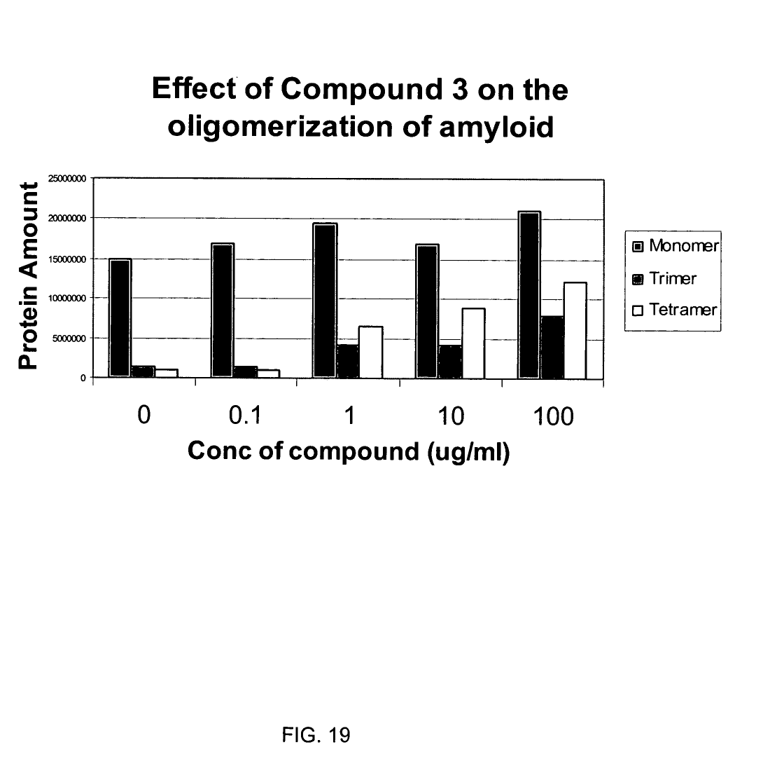

As shown in FIG. 18, incubation of amyloid peptides at 40 μM results in the formation of high molecular weight oligomers (fibrillogenesis), while incubation in the presence of compound 3 (lanes 3-7) results in a concentration dependent increase in the amount of monomer, as well as other low molecular weight oligomeric species, indicating that compound 3 inhibited fibrillogenesis of amyloid peptide. As in Example 8, disassociation of amyloid fibrils (lanes 8-12) was also shown by the increase of monomeric amyloid peptide in the presence of compound 3. these results were quantitated by scanning and densitometry as described above, and the results are shown in FIG. 19, which demonstrates the concentration dependent inhibition of amyloid peptide fibrillogenesis.

Unless defined otherwise, all technical and scientific terms used herein have the same meaning as commonly understood by one of ordinary skill in the art to which this disclosure belongs. Unless mentioned otherwise the techniques employed or contemplated herein are standard methodologies well known to one of ordinary skill in the art. The materials, methods and examples are illustrative only and not limiting. All numerical ranges in this specification are intended to be inclusive of their upper and lower limits.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

What is claimed is:

1. A method of treating an animal suffering from a disease or condition associated with pathogenic proteins, comprising administering to said mammal a pharmaceutical composition comprising a therapeutically effective amount of a compound of formula I 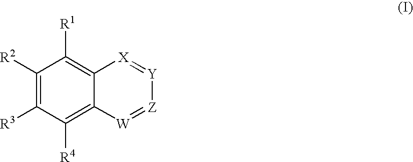
wherein R¹ is selected from the group consisting of H, Cl, Br, I, NO, NO₂, CN, C₁-C₆ alkyl, L¹-R¹⁸ or CF₃;
R² is selected from the group consisting of H, Cl, Br, I, NO, NO₂, CN, C₁-C₆ alkyl, L²-(C₁-C₆ alkyl) or CF₃;
R³ is selected from the group consisting of H, Cl, Br, I, NO, NO₂, CN, C₁-C₆ alkyl, L³-(C₁-C₆ alkyl) or CF₃;
R⁴ is selected from the group consisting of H, Cl, Br, I, NO, NO₂, CN, C₁-C₆ alkyl, L⁴-(C₁-C₆ alkyl), L⁴-aryl or CF₃, and each aryl is optionally substituted with 0-3 groups independently selected from F, Cl, Br, I, OR²⁹ NH₂, CO₂H, CF₃, CN, C₁-C₆ alkyl;
R⁵ is selected from the group consisting of H, L⁵-R³² or C(R⁷)₂R²⁸;
R⁶ is selected from the group consisting of H, Cl, Br, I, C₁-C₆ alkyl, L⁶-R⁸, CF₃ or aryl, and each aryl is optionally substituted with 0-3 groups independently selected from F, Cl, Br, I, OH, NH₂, CO₂H, CF₃, CN, C₁-C₆ alkyl;
R⁷ is independently selected from the group consisting of H, F, C₁-C₆ alkyl, OR⁹, SR¹⁰, S(═O)R¹¹, S(═O)₂R¹² or NR¹³R¹⁴;
R⁸ is selected from the group consisting of H or C₁-C₆ alkyl;
R⁹ is selected from the group consisting of H or C₁-C₆ alkyl;
R¹⁰ is selected from the group consisting of H or C₁-C₆ alkyl;
R¹¹ is C₁-C₆ alkyl;
R¹² is selected from the group consisting of OH, C₁-C₆ alkyl or O—(C₁-C₆ alkyl);
R¹³ is selected from the group consisting of H, C₁-C₆ alkyl or (C═O)—(C₁-C₆ alkyl);
R¹⁴ is selected from the group consisting of H, C₁-C₆ alkyl or (C═O)—(C₁-C₆ alkyl);
R¹⁵ is selected from the group consisting of H, C₁-C₆ alkyl or (C═O)—(C₁-C₆ alkyl);
R¹⁶ is selected from the group consisting of R⁶ or aryl, and each aryl is optionally substituted with 0-3 groups independently selected from F, Cl, Br, I, OH, NH₂, CO₂H, CF₃, CN, C₁-C₆ alkyl;
R¹⁷ is selected from the group consisting of H, C₁-C₆ alkyl or aryl, and each aryl is optionally substituted with 0-3 groups independently selected from F, Cl, Br, I, OH, NH₂, CO₂H, CF₃, CN, C₁-C₆ alkyl;
R¹⁸is H, C₁-C₆ alkyl or —(CH₂)ₙCR¹⁹R²⁰(CH₂)ₒ CR³⁰R³¹(CH₂)ₚ—R²¹;
R¹⁹ is selected from the group consisting of H, C₁-C₆ alkyl, OH or O—(C₁-C₆ alkyl);
R²⁰ is selected from the group consisting of H, C₁-C₆ alkyl, OH or O—(C₁-C₆ alkyl);
R²¹ is selected from the group consisting of H, C(═NH)NH₂, OR²², SR²³, S(═O)R²⁴, S(═O)₂R²⁵ or NR²⁶R²⁷;
R²² is selected from the group consisting of H or C₁-C₆ alkyl;
R²³ is selected from the group consisting of H or C₁-C₆ alkyl;
R²⁴ is C₁-C₆ alkyl;
R²⁵ is selected from the group consisting of OH, C₁-C₆ alkyl or O—(C₁-C₆ alkyl);
R²⁶ is selected from the group consisting of H, (CH₂)_{q}OH, C(═NH)NH₂, C₁-C₈ alkyl or (C═O)—(C₁-C₆ alkyl) where both R²⁶ and R²⁷ are not simultaneously OH and are not simultaneously C(═NH)NH₂;
R²⁷ is selected from the group consisting of H, (CH₂)ᵣOH, C(═NH)NH₂, C₁-C₈ alkyl or (C═O)—(C₁-C₆ alkyl) where both R²⁶ and R²⁷ are not simultaneously OH and are not simultaneously C(═NH)NH₂;
R²⁸ is selected from the group consisting of H, F and a cyclic amine selected from the following 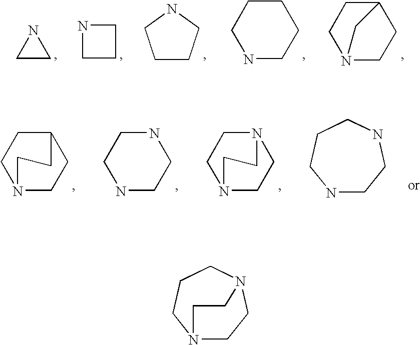
where the attachment point is at any available carbon or nitrogen atom, and all available carbon or nitrogen atoms are optionally substituted with a group independently selected from H, C₁-C₆ alkyl, C₁-C₆ alkenyl, C₁-C₆ alkynyl and (C═O)—(C₁-C₆ alkyl), where no more than two substitutions are not H;
R²⁹ is selected from the group consisting of H and C₁-C₆ alkyl;
R³⁰ is selected from the group consisting of H and C₁-C₆ alkyl;
R³¹ is selected from the group consisting of H and C₁-C₆ alkyl;
R³² is either C₁-C₆ alkyl or (CH₂)ₛR²¹;
L¹, L², L³, L⁴, L⁵ and L⁶ are independently selected from the group consisting of O, S, S(═O), S(═O)₂ and NR¹⁵;
W is N or CR⁵;
X is N or CR¹⁶;
Y is N or CR⁶;
Z is N or CR¹⁷;
n is an integer from 0-10;
o is an integer from 0-10;
p is an integer from 0-10;
n+o+p is an integer from 0-12;
q is an integer from 0-4;
r is an integer from 0-4; and
s is either 0 or an integer from 2-6; and
pharmaceutically acceptable salts, hydrates, and polymorphs thereof.

2. The method of claim 1, wherein the pharmaceutical composition comprises a therapeutically effective amount of a compound of formula II 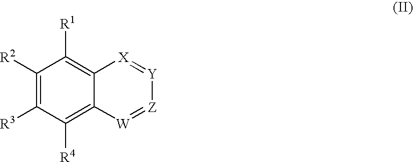
wherein R¹ is selected from the group consisting of H, Cl, Br, I, NO, NO₂, C₁-C₆ alkyl, L¹-R¹⁸ or CF₃;
R² is selected from the group consisting of H, Cl, Br, I, NO, NO₂, C₁-C₆ alkyl, L²-(C₁-C₆ alkyl) or CF₃;
R³ is selected from the group consisting of H, Cl, Br, I, NO, NO₂, C₁-C₆ alkyl, L³-(C₁-C₆ alkyl) or CF₃;
R⁴ is selected from the group consisting of H, Cl, Br, I, NO, NO₂, C₁-C₆ alkyl, L⁴-(C₁-C₆ alkyl), L⁴-aryl or CF₃ and each aryl is optionally substituted with 0-3 groups independently selected from F, Cl, Br, I, OR²⁹ NH₂, CO₂H, CF₃, C₁-C₆ alkyl;
R⁵ is H, L⁵-R³² or C(R⁷)₂R²⁸;
R⁶ is selected from the group consisting of H, Cl, Br, I, C₁-C₆ alkyl, L⁶-R⁸ or CF₃;
R⁷ is independently selected from the group consisting of H, F, C₁-C₆ alkyl, OR⁹, SR¹⁰, S(═O)R¹¹, S(═O)₂R¹² or NR¹³R¹⁴;
R⁸ is selected from the group consisting of H or C₁-C₆ alkyl;
R⁹ is selected from the group consisting of H or C₁-C₆ alkyl;
R¹⁰ is selected from the group consisting of H or C₁-C₆ alkyl;
R¹¹ is C₁-C₆ alkyl;
R¹² is selected from the group consisting of OH, C₁-C₆ alkyl or O—(C₁-C₆ alkyl);
R¹³ is selected from the group consisting of H, C₁-C₆ alkyl or (C═O)—(C₁-C₆ alkyl);
R¹⁴ is selected from the group consisting of H, C₁-C₆ alkyl or (C═O)—(C₁-C₆ alkyl);
R¹⁵ is selected from the group consisting of H, C₁-C₆ alkyl or (C═O)—(C₁-C₆ alkyl);
R¹⁷ is selected from the group consisting of H or C₁-C₆ alkyl;
R¹⁸ is H, C₁-C₆ alkyl or —(CH₂)ₙCR¹⁹R²⁰(CH₂)ₒ CR³⁰R³¹(CH₂)ₚ—R²¹;
R¹⁹ is selected from the group consisting of H, C₁-C₆ alkyl, OH or O—(C₁-C₆ alkyl);
R²⁰ is selected from the group consisting of H, C₁-C₆ alkyl, OH or O—(C₁-C₆ alkyl);
R²¹ is selected from the group consisting of H, C(═NH)NH₂, OR²², SR²³, S(═O)R²⁴, S(═O)₂R²⁵ or NR²⁶R²⁷;
R²² is selected from the group consisting of H or C₁-C₆ alkyl;
R²³ is selected from the group consisting of H or C₁-C₆ alkyl;
R²⁴ is C₁-C₆ alkyl;
R²⁵ is selected from the group consisting of OH, C₁-C₆ alkyl or O—(C₁-C₆ alkyl);
R²⁶ is selected from the group consisting of H, (CH₂)_{q}OH, C(═NH)NH₂, C₁-C₈ alkyl or (C═O)—(C₁-C₆ alkyl), where R²⁶ and R²⁷ are not both simultaneously OH or C(═NH)NH₂;
R²⁷ is selected from the group consisting of H, (CH₂)ᵣOH, C(═NH)NH₂, C₁-C₈ alkyl or (C═O)—(C₁-C₆ alkyl), where R²⁶ and R²⁷ are not both simultaneously OH or C(═NH)NH₂;
R²⁸ is selected from the group consisting of H, F, and a cyclic amine selected from the following 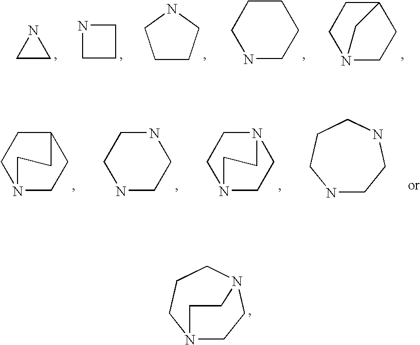
where the attachment point is at any available carbon or nitrogen atom, and all available carbon or nitrogen atoms are additionally substituted with groups independently selected from H, C₁-C₆ alkyl, C₁-C₆ alkenyl, C₁-C₆ alkynyl or (C═O)—(C₁-C₆ alkyl), where no more than two substitutions are not H;
R²⁹ is selected from the group consisting of H or C₁-C₆ alkyl;
R³⁰ is selected from the group consisting of H or C₁-C₆ alkyl;
R³¹ is selected from the group consisting of H or C₁-C₆ alkyl;
R³² is either C₁-C₆ alkyl or (CH₂)ₛR²¹;
L¹, L², L³, L⁴, L⁵ and L⁶ are independently selected from the group consisting of O, S, S(═O), S(═O)₂ or NR¹⁵;
W is N or CR⁵;
X is N or CR⁶;
Y is N or CR⁶;
Z is N or CR¹⁷;
n is an integer from 0-10;
o is an integer from 0-10;
p is an integer from 0-10;
n+o+p is an integer from 0-12;
q is an integer from 0-4;
r is an integer from 0-4;
s is either 0 or an integer from 2-6; and
pharmaceutically acceptable salts, hydrates, and polymorphs thereof.

3. The method of claim 1, wherein the pharmaceutical composition comprises a therapeutically effective amount of a compound of formula III 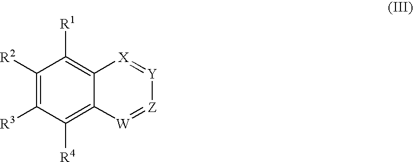
wherein R¹ is selected from the group consisting of H, Cl, Br, I, NO, NO₂, C₁-C₆ alkyl, L¹-R¹⁸ or CF₃;
R² is selected from the group consisting of H, Cl, Br, I, NO, NO₂, C₁-C₆ alkyl, L²-(C₁-C₆ alkyl) or CF₃;
R³ is selected from the group consisting of H, Cl, Br, I, NO, NO₂, C₁-C₆ alkyl, L³-(C₁-C₆ alkyl) or CF₃;
R⁴ is selected from the group consisting of H, Cl, Br, I, NO, NO₂, C₁-C₆ alkyl, L⁴-(C₁-C₆ alkyl), L⁴-aryl or CF₃ and each aryl is optionally substituted with 0-3 groups independently selected from F, Cl, Br, I, OR²⁹ NH₂, CO₂H, CF₃, C₁-C₆ alkyl;
R¹ is H, L⁵-R³² or C(R⁷)₂R²⁸;
R⁶ is selected from the group consisting of H, Cl, Br, I, C₁-C₆ alkyl, L⁶-R⁸ or CF₃;
R⁷ is independently selected from the group consisting of H, F, C₁-C₆ alkyl, OR⁹, SR¹⁰ or NR¹³R¹⁴;
R⁸ is selected from the group consisting of H or C₁-C₆ alkyl;
R⁹ is selected from the group consisting of H or C₁-C₆ alkyl;
R¹⁰ is selected from the group consisting of H or C₁-C₆ alkyl;
R¹³ is selected from the group consisting of H, C₁-C₆ alkyl or (C═O)—(C₁-C₆ alkyl);
R¹⁴ is selected from the group consisting of H, C₁-C₆ alkyl or (C═O)—(C₁-C₆ alkyl);
R¹⁵ is selected from the group consisting of H, C₁-C₆ alkyl or (C═O)—(C₁-C₆ alkyl);
R¹⁷ is selected from the group consisting of H or C₁-C₆ alkyl;
R¹⁸ is H, C₁-C₆ alkyl or —(CH₂)ₙCR¹⁹R²⁰(CH₂)ₒ CR³⁰R³¹(CH₂)ₚ—R²;
R¹⁹ is selected from the group consisting of H, C₁-C₆ alkyl, OH or O—(C₁-C₆ alkyl);
R²⁰ is selected from the group consisting of H, C₁-C₆ alkyl, OH or O—(C₁-C₆ alkyl);
R²¹ is selected from the group consisting of H, C(═NH)NH₂, OR²², SR²³ or NR²⁶R²⁷;
R²² is selected from the group consisting of H or C₁-C₆ alkyl;
R²³ is selected from the group consisting of H or C₁-C₆ alkyl;
R²⁶ is selected from the group consisting of H, (CH₂)_{q}OH, C(═NH)NH₂, C₁-C₈ alkyl or (C═O)—(C₁-C₆ alkyl) where R²⁶ and R²⁷ are not both simultaneously OH or C(═NH)NH₂;
R²⁷ is selected from the group consisting of H, (CH₂)ᵣOH, C(═NH)NH₂, C₁-C₈ alkyl or (C═O)—(C₁-C₆ alkyl), where R²⁶ and R²⁷ are not both simultaneously OH or C(═NH)NH₂;
R²⁸ is selected from the group consisting of H, F and a cyclic amine selected from the following 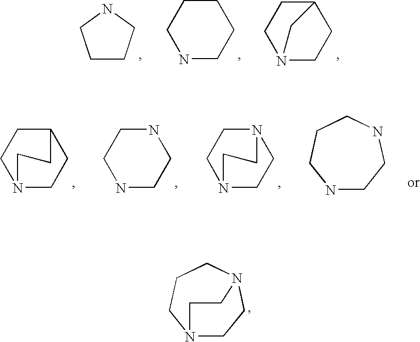
where the attachment point is at any available carbon or nitrogen atom, and all available carbon or nitrogen atoms are additionally substituted with groups independently selected from H, C₁-C₆ alkyl, C₁-C₆ alkenyl, C₁-C₆ alkynyl or (C═O)—(C₁-C₆ alkyl), where no more than two substitutions are not H;
R²⁹ is selected from the group consisting of H or C₁-C₆ alkyl;
R³⁰ is selected from the group consisting of H or C₁-C₆ alkyl;
R³¹ is selected from the group consisting of H or C₁-C₆ alkyl;
R³² is either C₁-C₆ alkyl or (CH₂)ₛR²¹;
L¹, L², L³, L⁴, L⁵ and L⁶ are independently selected from the group consisting of O, S or NR¹⁵;
W is N or CR⁵;
X is N or CR⁶;
Y is N or CR⁶;
Z is N or CR¹⁷;
n is an integer from 0-10;
o is an integer from 0-10;
p is an integer from 0-10;
n+o+p is an integer from 0-12;
q is an integer from 0-4;
r is an integer from 0-4;
s is either 0 or an integer from 2-6; and
pharmaceutically acceptable salts, hydrates, and polymorphs thereof.

4. The method of claim 1, wherein the pharmaceutical composition comprises a therapeutically effective amount of a compound of formula IV 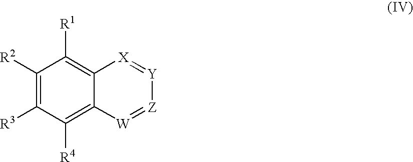
wherein R¹ is selected from the group consisting of H, C₁-C₆ alkyl, L¹-R¹⁸ or CF₃;
R² is selected from the group consisting of H, C₁-C₆ alkyl, L²-(C₁-C₆ alkyl) or CF₃;
R³ is selected from the group consisting of H, C₁-C₆ alkyl, L³-(C₁-C₆ alkyl) or CF₃;
R⁴ is selected from the group consisting of H, C₁-C₆ alkyl, L⁴-(C₁-C₆ alkyl) or CF₃;
R⁵ is C(R⁷)₂R²⁸;
R⁶ is selected from the group consisting of H, C₁-C₆ alkyl, L⁶-R⁸ or CF₃;
R⁷ is independently selected from the group consisting of H or OR⁹;
R⁸ is C₁-C₆ alkyl;
R⁹ is selected from the group consisting of H or C₁-C₆ alkyl;
R¹⁵ is selected from the group consisting of H, C₁-C₆ alkyl or (C═O)—(C₁-C₆ alkyl;
R¹⁷ is selected from the group consisting of H or C₁-C₆ alkyl;
R¹⁸ is H or C₁-C₆ alkyl;
R²⁸ is selected from a cyclic amine selected from the following 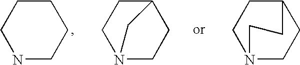
where the attachment point is at any available carbon or nitrogen atom, and all available carbon or nitrogen atoms are additionally substituted with groups independently selected from H, C₁-C₆ alkyl, C₁-C₆ alkenyl, C₁-C₆ alkynyl or (C═O)—(C₁-C₆ alkyl), where no more than two substitutions are not H;
L¹, L², L³, L⁴ and L⁶ are independently selected from the group consisting of O, S or NR¹⁵;
W is CR⁵;
X is N;
Y is CR⁶;
Z is CR¹⁷; and
pharmaceutically acceptable salts, hydrates, and polymorphs thereof.

5. The method of claim 1, wherein the pharmaceutical composition comprises a therapeutically effective amount of a compound of formula V 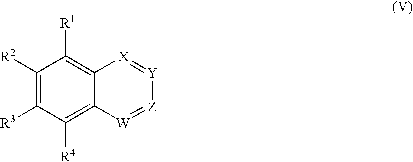
wherein R¹ is selected from the group consisting of H, C₁-C₆ alkyl, L¹-R¹⁸ or CF₃;
R² is selected from the group consisting of H, C₁-C₆ alkyl, L²-(C₁-C₆ alkyl) or CF₃;
R³ is selected from the group consisting of H, C₁-C₆ alkyl, L³-(C₁-C₆ alkyl) or CF₃;
R⁴ is L⁴-aryl and each aryl is optionally substituted with 0-3 groups independently selected from F, Cl, Br, I, OR²⁹ NH₂, CO₂H, CF₃, C₁-C₆ alkyl;
R⁵ is H, L⁵-R³² or C(R⁷)₂R²⁸;
R⁶ is selected from the group consisting of H, C₁-C₆ alkyl, L⁶-R⁸ or CF₃;
R⁷ is H;
R⁸ is C₁-C₆ alkyl;
R¹⁵ is selected from the group consisting of H, C₁-C₆ alkyl or (C═O)—(C₁-C₆ alkyl);
R¹⁷ is H;
R¹⁸ is —(CH₂)ₙCR¹⁹R²⁰(CH₂)ₒ CR³⁰R³¹ (CH₂)ₚ—R²¹;
R¹⁹ is H or C₁-C₆ alkyl;
R²⁰ is H or C₁-C₆ alkyl;
R²¹ is NR²⁶R²⁷;
R²⁶ is H or C₁-C₈ alkyl;
R²⁷ is H or C₁-C₈ alkyl;
R²⁸ is H;
R²⁹ is selected from the group consisting of H or C₁-C₆ alkyl;
R³⁰ is selected from the group consisting of H or C₁-C₆ alkyl;
R³¹ is selected from the group consisting of H or C₁-C₆ alkyl;
R³² is C₁-C₆ alkyl;
L¹, L², L³, L⁴, L⁵ and L⁶ are independently selected from the group consisting of O, S or NR¹⁵
W is CR⁵;
X is N;
Y is CR⁶; is CR¹⁷;
n is an integer from 0-5;
o is an integer from 0-5;
p is an integer from 0-5;
n+o+p is an integer from 0-5; and
pharmaceutically acceptable salts, hydrates, and polymorphs thereof.

6. The method of claim 1, wherein said animal is a farm animal, a companion animal, a zoo animal, a laboratory animal, a wild animal or a game animal.

7. The method of claim 6, wherein the farm animal is a cow, pig, sheep, chicken or goat.

8. The method of claim 1, wherein the animal is a mammal, including a human patient.

9. The method of claim 1, wherein the therapeutically effective amount is from about 0.5 to about 10,000 mg/day/75 kg of body weight.

10. The method of claim 1, wherein the composition is administered topically, transdermally, transmucosally, orally, parenterally, or via inhalation.

11. The method of claim 1, wherein the animal is suffering from a disease selected from Creutzfeldt-Jakob disease, scrapie, transmissible spongiform encephalopathy (TSE), Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy, AIDS dementia, systemic senile amyloidosis, prion disease, Gerstmann-Straussler-Scheinker syndrome, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, or lysozyme-associated hereditary systemic amyloidosis.

12. The method of claim 11, wherein said pharmaceutical composition further comprises at least one additional pharmaceutically active agent.

13. The method of claim 12, wherein the additional pharmaceutically active agent is selected from one or more of a cholinomimetic, a cholinesterase inhibitor, a secretase inhibitor, an NMDA receptor antagonist, an amyloid binding protein, insulin or an insulin mimetic, an insulin sensitizer, a PPARγ receptor antagonist, colchicine, an anti-inflammatory agent, GSK-3 inhibitor, levodopa, dopamine receptor agonist, catechol —O-methyl transferase inhibitor, MAO-B inhibitor, muscarinic receptor antagonist, diphenylhydramine HCl, riluzol, baclofen, tizanidine, clonazepam, or dantroline.

14. The method of claim 1, wherein the pharmaceutical composition comprises tafenoquine, compound 3, compound 8, or mefloquin, and pharmaceutically acceptable salts, hydrates or polymorphs thereof.

15. A composition for treating livestock having a disease or condition associated with pathogenic proteins, wherein said composition comprises: livestock feed; and a compound of formula I.

16. The composition of claim 15, wherein the compound is selected from tafenoquine, compound 3, compound 8, mefloquin, and pharmaceutically acceptable salts, hydrates or polymorphs thereof.

17. A method for clearing pathogenic proteins from livestock, said method comprising:
(a) feeding the livestock an effective amount of the composition of claim 14; and
(b) repeatedly providing composition to livestock over a therapeutically effective period of time.

18. A method of treating a disease characterized by pathogenic protein formation, the method comprising administering a therapeutically effective amount of a compound of formula I.

19. A method of preventing disease in a mammal that has been exposed to a protease resistant prion protein, the method comprising administering to said mammal a therapeutically effective amount of a compound of formula I.

20. A method of preventing disease in an animal that is in danger of being exposed to a protease resistant prion protein, the method comprising administering to said animal a therapeutically effective amount of a compound of formula I.

21. A method of treating disease in an animal that is exhibiting signs, symptoms or laboratory evidence of a transmissible spongiform encephalopathy, the method comprising administering to said animal a therapeutically effective amount of a compound of formula I.

22. A method for reducing or retarding degeneration in a cell population of a tissue or organ subject to damage in an amyloid disease, comprising exposing the cell population to a therapeutically effective amount of a compound of formula I.

23. A method for reducing necrosis or apoptosis in a cell population of a tissue or organ subject to necrotic or apoptotic damage in an amyloid disease, comprising exposing the cell population to a therapeutically effective amount of a compound of formula I.

24. The method of claim 23, wherein said cell population comprises pancreatic islet cells, neurons, glia, endothelial cells, or endocrine cells.

25. A method of blocking amyloid toxicity in cells, said method comprising contacting said cells with an effective amount of at least one compound of formula I.

26. The method of claim 24, wherein said amyloid toxicity is selected from amyloid beta peptide toxicity, amyloid prion protein toxicity, human amylin toxicity, amyloid A protein toxicity, transthyretin toxicity, and AL amyloid toxicity.

27. A method for reducing or retarding neurodegeneration in a cell population comprising neurons which have been exposed to an amount of a stimulus sufficient to produce at least partially protease resistant proteins resulting in neurodegeneration, the method comprising: exposing the cell population to a therapeutically effective amount of a compound of formula I.

28. A method for inhibition of neuronal cell death in a cell population that is exposed to a protease resistant prion protein stimulus, the method comprising: administering a therapeutically effective amount of a compound of formula I to the cell population.

29. A method of treating a product derived from a human source to reduce the risk of transmission of an infectious pathogenic protein, the method comprising: combining a compound of formula I with said product.

30. The method of claim 29, wherein said product derived from a human source is a product selected from organs, tissue, blood, hormones or related blood derived products.

31. A method for slowing, arresting, or reversing the development of a neurodegenerative disease in a human patient, the method comprising: administering a therapeutically effective amount of a compound of formula I to the patient to inhibit progression of the disease over a therapeutically effective period of time.

32. A kit for use in treating disease or disorder associated with pathogenic proteins, the kit comprising:
a container containing a pharmaceutical composition comprising a therapeutically effective amount of a compound of formula I, and
instructions comprising guidelines for administration of the composition according the method of claim 1.

32. The kit of claim 32, wherein the kit further comprises an additional pharmaceutical composition comprising an active agent useful in treating the disease or disorder.

33. The kit of claim 32, wherein said instructions comprise an indication of the quantities of the composition to be administered.

34. A pharmaceutical composition for treating a mammal suffering from a disease or condition associated with pathogenic proteins, comprising a therapeutically effective amount of a compound of formula I 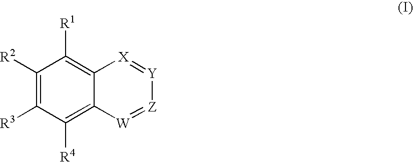
wherein R¹ is selected from H, Cl, Br, I, NO, NO₂, CN, C₁-C₆ alkyl, L¹-R¹⁸ or CF₃;
R² is selected from H, Cl, Br, I, NO, NO₂, CN, C₁-C₆ alkyl, L²-(C₁-C₆ alkyl) or CF₃;
R³ is selected from H, Cl, Br, I, NO, NO₂, CN, C₁-C₆ alkyl, L³-(C₁-C₆ alkyl) or CF₃;
R⁴ is selected from H, Cl, Br, I, NO, NO₂, CN, C₁-C₆ alkyl, L⁴-(C₁-C₆ alkyl), L⁴-aryl or CF₃, and each aryl is optionally substituted with 0-3 groups independently selected from F, Cl, Br, I, OR²⁹ NH₂, CO₂H, CF₃, CN, C₁-C₆ alkyl;
R⁵ is selected from H, L⁵-R³² or C(R⁷)₂R²⁸;
R⁶ is selected from H, Cl, Br, I, C₁-C₆ alkyl, L⁶-R⁸, CF₃ or aryl, and each aryl is optionally substituted with 0-3 groups independently selected from F, Cl, Br, I, OH, NH₂, CO₂H, CF₃, CN, C₁-C₆ alkyl;
R⁷ is independently selected from H, F, C₁-C₆ alkyl, OR⁹, SR¹⁰, S(═O)R¹¹, S(═O)₂R¹² or NR¹³R¹⁴;
R⁸ is selected from H or C₁-C₆ alkyl;
R⁹ is selected from H or C₁-C₆ alkyl;
R¹⁰ is selected from H or C₁-C₆ alkyl;
R¹¹ is C₁-C₆ alkyl;
R¹² is selected from OH, C₁-C₆ alkyl or O—(C₁-C₆ alkyl);
R¹³ is selected from H, C₁-C₆ alkyl or (C═O)—(C₁-C₆ alkyl);
R¹⁴ is selected from H, C₁-C₆ alkyl or (C═O)—(C₁-C₆ alkyl);
R¹⁵ is selected from H, C₁-C₆ alkyl or (C═O)—(C₁-C₆ alkyl);
R¹⁶ is selected from R⁶ or aryl, and each aryl is optionally substituted with 0-3 groups independently selected from F, Cl, Br, I, OH, NH₂, CO₂H, CF₃, CN, C₁-C₆ alkyl;
R¹⁷ is selected from H, C₁-C₆ alkyl or aryl, and each aryl is optionally substituted with 0-3 groups independently selected from F. Cl, Br, I, OH, NH₂, CO₂H, CF₃, CN, C₁-C₆ alkyl;
R¹⁸ is H, C₁-C₆ alkyl or —(CH₂)ₙCR¹⁹R²⁰(CH₂)ₒ CR³⁰R³¹(CH₂)ₚ—R²¹;
R¹⁹ is selected from H, C₁-C₆ alkyl, OH or O—(C₁-C₆ alkyl);
R²⁰ is selected from H, C₁-C₆ alkyl, OH or O—(C₁-C₆ alkyl);
R²¹ is selected from H, C(═NH)NH₂, OR²², SR²³, S(═O)R²⁴, S(═O)₂R²⁵ or NR²⁶R²⁷.
R²² is selected from H or C₁-C₆ alkyl;
R²³ is selected from H or C₁-C₆ alkyl;
R²⁴ is C₁-C₆ alkyl;
R²⁵ is selected from OH, C₁-C₆ alkyl or O—(C₁-C₆ alkyl);
R²⁶ is selected from H, (CH₂)_{q}OH, C(═NH)NH₂, C₁-C₈ alkyl or (C═O)—(C₁-C₆ alkyl) where both R²⁶ and R²⁷ are not simultaneously OH and are not simultaneously C(═NH)NH₂;
R²⁷ is selected from H, (CH₂)ᵣOH, C(═NH)NH₂, C₁-C₈ alkyl or (C═O)—(C₁-C₆ alkyl) where both R²⁶ and R²⁷ are not simultaneously OH and are not simultaneously C(═NH)NH₂;
R²⁸ is selected from H, F and a cyclic amine consisting of the following structures 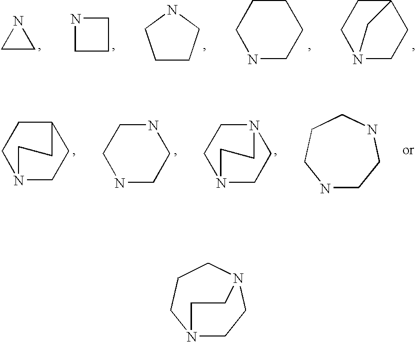
where the attachment point is at any available carbon or nitrogen atom, and all available carbon or nitrogen atoms are optionally substituted with a group independently selected from H, C₁-C₆ alkyl, C₁-C₆ alkenyl, C₁-C₆ alkynyl and (C═O)—(C₁-C₆ alkyl), where no more than two substitutions are not H;
R²⁹ is selected from H and C₁-C₆ alkyl;
R³⁰ is selected from H and C₁-C₆ alkyl;
R³¹ is selected from H and C₁-C₆ alkyl;
R³² is either C₁-C₆ alkyl or (CH₂)ₛR²¹;
L¹, L², L³, L⁴, L⁵ and L⁶ are independently selected from the group consisting of O, S, S(═O), S(═O)₂ and NR¹⁵;
W is N or CR⁵;
X is N or CR¹⁶;
Y is N or CR⁶;
Z is N or CR¹⁷;
n is an integer from 0-10;
o is an integer from 0-10;
p is an integer from 0-10;
n+o+p is an integer from 0-12;
q is an integer from 0-4;
r is an integer from 0-4; and
s is either 0 or an integer from 2-6; and
pharmaceutically acceptable salts, hydrates, and polymorphs thereof; and optionally a pharmaceutically acceptable carrier.

35. The composition of claim 34, wherein the disease or condition associated with pathogenic proteins is Creutzfeldt-Jakob disease, scrapie, transmissible spongiform encephalopathy (TSE), Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy, AIDS dementia, systemic senile amyloidosis, prion disease, Gerstmann-Straussler-Scheinker syndrome, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, or lysozyme-associated hereditary systemic amyloidosis.

36. The composition of claim 34, further comprising at least one additional pharmaceutically active agent.

37. The composition of claim 36, wherein the additional pharmaceutically active agent is selected from one or more of a cholinomimetic, a cholinesterase inhibitor, a secretase inhibitor, an NMDA receptor antagonist, an amyloid binding protein, insulin or an insulin mimetic, an insulin sensitizer, a PPARγ receptor antagonist, colchicine, an anti-inflammatory agent, GSK-3 inhibitor, levodopa, dopamine receptor agonist, catechol —O-methyl transferase inhibitor, MAO-B inhibitor, muscarinic receptor antagonist, diphenylhydramine HCl, riluzol, baclofen, tizanidine, clonazepam, or dantroline.
